(19) United States
(12) Patent Application Publication
(10) Pub. No.: US 2022/0402988 A1
(43) Pub. Date: Dec. 22, 2022

(54) INTERLEUKIN 15 FUSION PROTEINS AND PRODRUGS, AND COMPOSITIONS AND METHODS THEREOF

(71) Applicant: IMMUNE TARGETING INC., Dallas, TX (US)

(72) Inventor: Yang Wang, Dallas, TX (US)

(21) Appl. No.: 17/777,088

(22) PCT Filed: Dec. 4, 2020
(86) PCT No.: PCT/US20/63214

§ 371 Date: May 16, 2022

Related U.S. Application Data
(60) Provisional application No. 62/944,203, filed on Dec. 5, 2019.

(51) Int. Cl. C07K 14/54; A61P 35/00; C12N 15/62

(52) U.S. Cl. CPC C07K 14/5443; A61P 35/00; C12N 15/62; C07K 2319/30; C07K 2319/50

(57) ABSTRACT

The invention provides novel fusion proteins and prodrugs of Interleukin 15, and compositions and methods of preparation thereof, that are useful in treating various diseases and disorders (e.g., hyperplasia, solid tumor or hematopoietic malignancy).

### PRIORITY CLAIMS AND RELATED APPLICATIONS

This application claims the benefit to U.S. Provisional Application Ser. No. 62/944,203, filed Dec. 5, 2020, the entire content of which is incorporated herein by reference for all purposes.

### TECHNICAL FIELD OF THE INVENTION

The invention generally relates to novel fusion proteins and therapeutic uses thereof. More particularly, the invention provides novel fusion proteins and prodrugs of Interleukin 15 (IL15 or IL-15), and compositions and methods of preparation thereof, that are useful in treating various diseases and disorders (e.g., hyperplasia, solid tumor or hematopoietic malignancy).

### BACKGROUND OF THE INVENTION

Most tumor immunotherapies focus on blocking immune inhibitory signals or activating immune stimulatory pathways. However, several issues remain and hinder the clinical benefits, including inducing severe treatment-associated organ toxicity and poor tumor control due to off-tumor-targeted delivery. Interleukin 2 (IL2), one pleiotropic cytokine which could expand T cells and NK cells, was one of the earliest first FDA approved immunotherapy drugs for metastasis melanoma and renal cell cancer. (Rosenberg, et al 2014 J Immunol 192(12): 5451-5458.) However, IL2 has not been widely used in clinic, due to activation of immune inhibitory regulatory T cell (Tregs) and occasional sever toxicity result from activation of vascular endothelium. (Kolitz, et al. 2014 Cancer 120(7): 1010-1017; Sim, et al. 2014 J Clin Invest 124(1): 99-110.)

IL15 receptor (IL15R) belongs to the super family of hemopoietic system. The heterotrimeric IL15R comprises α, β (CD122) and γ (CD132, common gamma chain, γc) subunits. The β subunit (IL15Rβ) is shared with the IL2 receptor. Human IL15Rα belongs to type I transmembrane protein. Both IL2Rα and IL15Rα contain a conserved sushi domain. IL15 would have some similar functions as IL2, such as promoting the proliferation of T and NK cells. (Thomas et al. 2006 J of Immunology 177:6072-6080.)

Interleukin 15 (IL15), a 14-15 kDa glycoprotein, is a soluble cytokine that was first discovered in 1994. (Grabstein et al. 1994 Science 264:965-8.) Similar to IL2, IL15 belongs to the family of the four-helix-bundle cytokines. The human IL15 gene was mapped to chromosome 4 region q25-35. Mature IL15 consists of 112 amino acid and containing 3 N-glycosylation sites. Expression of IL15 is stimulated by cytokines such as granulocyte-macrophage colony-stimulating factor (GM-CSF), interferons, and agonists of Toll-like receptors (TLRs). (Marek et al. 2011 Cytokine & Growth Factor Reviews 22:99-108.) Various side-effects have been associated with IL15 therapy, such as induction of cytokine cascade, including TNFα, IL1, IL6, GM-CSF and pro-inflammatory cytokines; promotion of proliferation, survival, and metastasis of some tumor cells; activation of autoimmune T cells and participation of autoimmune diseases; induction of coronary heart disease; and induction the expression of inhibitory molecules PD1/PDL1.

There have been several forms of IL15 under research or development, including the IL-15:IL15RαSu/Fc soluble complex and IL-15-IL15Rα-Fc fusion protein. Most studies of these proteins identified the expansion of peripheral NK and CD8⁺ T cells as activity index, and tested preferentially in metastasis tumor or hematological cancer. (Jakobisiak, et al. 2011 Cytokine Growth Factor Rev 22(2): 99-108.)

However, the expansion of peripheral NK cells and IFN-γ contribute to systemic toxicity, raising the concern that administrative dose of IL-15 should be carefully limited during tumor therapy. (Guo, et al. 2015 J Immunol 195(5): 2353-2364.) These concerns highlight the needs to develop new IL15 approaches to specifically activate NK or T cells locally within tumor while avoiding the expansion of peripheral NK cells.

Recent studies showed that IL-15 within the tumor microenvironment is crucial for the optimal antitumor response. IL-15 is a component of the inflammatory milieu within tumor tissue, which was required for establishing normal number of CD8⁺ T cells and NK cells. Loss of IL15 within colorectal tumors correlated with decreased T cell proliferation, higher tumor recurrence, and decreased patient survival. (Curran, et al. 2013 J Exp Med 210(4): 743-755; Mlecnik, et al. 2014 Sci Transl Med 6(228): 228ra237; Santana Carrero, et al. 2019 Proc Natl Acad Sci USA 116(2): 599-608.) However, administration of exogenous IL-15 is not effective in the solid tumor therapy, possibly due to the lack of efficient tumor-targeted delivery method for IL-15. By now, most studies focus on prolong the half-life of IL-15, or increase its affinity with receptor.

The therapeutics and methods currently available for e.g., hyperplasia, solid tumor or hematopoietic malignancy are inadequate.

There remains an urgent and ongoing need for novel and improved efficacy towards such diseases and conditions with reduced side effects.

### SUMMARY OF THE INVENTION

The invention is based in part on the surprising discovery of novel fusion proteins and therapeutic uses thereof. Novel fusion proteins of IL15 and prodrugs thereof, compositions and methods of preparation thereof, are disclosed herein which are useful in treating various diseases and disorders, e.g., hyperplasia, solid tumor or hematopoietic malignancy.

A significant feature of an aspect of the present invention is the utilization of a shorter domain of IL15 receptor β, as opposed to the whole extra cellular domain of IL15 receptor β (27aa-240aa), linked via a MMP cleavable linker to super IL15 (IL15-Rα) in blocking its activity in circulation. Rb is cleaved off inside tumor tissues with elevated MMP enzymes resulting in removal of blockage and reactivation of sIL15 inside a tumor microenvironment. This approach led to an improved antitumor activity without increasing its toxicity profile. As disclosed herein, various short forms of IL15 receptor β were studied, for example, domain 1 (D1) corresponding to 27aa-125aa (or 27aa-128aa), along with various release and activation mechanisms through cleavable flexible linkers.

In one aspect, the invention generally relates to a fusion protein or fusion protein complex (A), being a homodimeric complex and comprising: a first structural unit: a whole or a subunit domain of the interleukin 15 (IL15) receptor-α; a second structural unit: an active IL15; a third structural unit located at the C-terminus of the fusion protein: an antibody Fc fragment; a fourth structural unit located at the N-terminus of the fusion protein: a whole or a subunit domain of the IL15 receptor β linked to the first, second or third structure unit via a short cleavable linker (L2) or long cleavable linker (L2L); and one or more connecting segments or ligation fragments as flexible non-cleavable linkers (L1) for connecting the different units.

In another aspect, the invention generally relates to a fusion protein or fusion protein complex (B), being a homodimeric complex and comprising: a first fragment (Fragment No. 1) and a second fragment (Fragment No. 2), bound via disulfide bonds. Fragment No. 1 comprises: a first structural unit: a whole or a subunit domain of the interleukin 15 (IL15) receptor-α, and a fifth structural unit: a constant region of light chain (CL). Fragment No. 2 comprises: a second structural unit: an active IL15, a third structural unit located at the C-terminus of the fusion protein: an antibody Fc fragment, a fourth structural unit located at the N-terminus of the fusion protein: a whole or a subunit domain of the IL15 receptor β; and linked with a short cleavable linker (L2) or long cleavable linker (L2L), a sixth structural unit: a constant region of heavy chain (CH), and one or more connecting segments or ligation fragments as flexible non-cleavable linkers (L1) for connecting the different units. Two Fragment No. 1 and two Fragment No. 2 are joined together to form a complex via disulfide bonds between CH and CL and between two Fc's.

In yet another aspect, the invention generally relates to homodimeric pro proteins or fusion protein complexes, comprising a fusion protein disclosed herein.

In yet another aspect, the invention generally relates to a prodrug comprising a fusion protein or fusion protein complex disclosed herein.

In yet another aspect, the invention generally relates to a substantially purified fusion protein or fusion protein complex, or prodrug thereof, disclosed herein.

In yet another aspect, the invention generally relates to a polynucleotide encoding a fusion protein or fusion protein complex, or prodrug thereof, disclosed herein.

In yet another aspect, the invention generally relates to an expression vector comprising a polynucleotide disclosed herein.

In yet another aspect, the invention generally relates to a pharmaceutical composition comprising a fusion protein or fusion protein complex, or prodrug thereof, disclosed herein.

In yet another aspect, the invention generally relates to a method for treating a disease or condition. The method comprises administering to a patient in need thereof a therapeutically effective amount of a fusion protein or fusion protein complex, or prodrug thereof, or a pharmaceutical composition disclosed herein, wherein the disease or condition is selected from hyperplasia, solid tumor or hematopoietic malignancy.

In yet another aspect, the invention generally relates to use of a fusion protein or fusion protein complex, or prodrug thereof, disclosed herein for treating or reducing a disease or disorder (e.g., hyperplasia, solid tumor or hematopoietic malignancy).

In yet another aspect, the invention generally relates to use of a polynucleotide encoding a protein, such as a fusion protein or a fragment thereof, disclosed herein for treating or reducing a disease or disorder (e.g., hyperplasia, solid tumor or hematopoietic malignancy).

In yet another aspect, the invention generally relates to use of a fusion protein or fusion protein complex, or prodrug thereof, disclosed herein and a pharmaceutically acceptable excipient, carrier, or diluent, in preparation of a medicament for treating or reducing a disease or disorder (e.g., hyperplasia, solid tumor or hematopoietic malignancy).

In yet another aspect, the invention generally relates to a cell line comprising a polynucleotide encoding a fusion protein or fusion protein complex, or prodrug thereof, disclosed herein.

In yet another aspect, the invention generally relates to a method for making a protein, comprising culturing the cell line. In certain embodiments, the method further comprises purifying or isolating a produced protein, such as a fusion protein or fusion protein complex, or prodrug thereof disclosed herein.

In yet another aspect, the invention generally relates to a method for making a protein. The method comprises: providing an expression vector encoding a fusion protein or fusion protein complex, or prodrug thereof disclosed herein; introducing into a host cell the expression vector; culturing the host cell in media under conditions sufficient to express the protein; and purifying the protein from the host cell or media.

### BRIEF DESCRIPTION OF THE DRAWINGS

FIG. 1. Poor tumor control and severe toxicity after systemic delivery of IL15-RA-Fc. (a) The activity of indicated proteins were detected by CTLL2 proliferation assay. (b) Balb/c mice (n=5) were inoculated with 3×10⁶ A20 cells. After tumor established (about 40 mm³), mice were treated with 0.2 μmol sIL15-Fc or sIL15-his by i.v. injection on days 8 and 11. (c), (d) C57BL/6 mice were inoculated with 5×10⁵ MC38 cells. After tumor established (about 40 mm³), mice were treated with 0.8 μmol sIL-15-Fc (n=16) by i.v. injection on days 8 and 11. The body weight (c) and mice survival (d) were monitored. (e)-(g) MC38 tumor-bearing mice (n=5) were treated with 0.8 μmol sIL-15-Fc by i.p. injection on days 8 and 11. For NK cell depletion, mice were i.p. injected with 20 μL of anti-Asialo GM1 antibody on days 8 and 11. On day 5 after first anti-Asialo GM1 treatment, the numbers of indicated cell subsets in peripheral blood were counted by FACS (e). The body weight change (f) and survival (g) of the mice were monitored.

FIG. 2. Engineering a tumor-conditional pro-IL-15. (a) MC38 tumor bearing mice (n=4 to 5) were treated with 0.2 μmol sIL-15-Fc by i.t. or i.v. injection on days 11 and 14. (b) Schematic representation of pro-IL-15 protein. (c),(d) pro-IL-15 was incubated with pre-activated MMP14 at 37 degree in vitro for 12 hours. The proteins cleavage was identified by reduced SDS-PAGE (c) and released activity of pro-IL-15 was detected by CTLL2 proliferation assay (d). e MC38 tumor bearing mice were treated with 0.25 μmol pro-IL-15(Rβ) or pro-IL-15(RβD1) by i.p. injection on days 11, 14 and 18 respectively.

FIG. 3. Pro-IL-15 avoid peripheral NK expansion mediated toxicity. (a),(b) MC38 tumor bearing mice were treated with PBS(n=6), 0.8 μmol sIL-15 (n=12), or pro-IL-15 (n=6) by i.v. injection on days 8 and 11. The body weight change of mice (a) and survival (b) were monitored. (c),(d) MC38 tumor bearing mice were treated same as a and b. The level of ALT (c) and AST (d) in the peripheral serum was quantified at day 1 and day 4 after first IL-15 treatment. (e),(f) MC38 tumor-bearing mice were treated with 0.5 μmol sIL-15-Fc or pro-IL-15 on days 8 and 11 after tumor inoculation. Blood was collected at 24 hours after first treatment. Cytokine levels in the serum were measured by cytometric bead array (e). The lymphocytes expansion in the peripheral blood was tested by FACS on day 5 after first treatment (f).

FIG. 4. Pro-IL-15D1 preserved antitumor activity. (a) Pro-IL-15 (D1) protein was conjugated with Cy5.5-maleimide. 25 μg of Cy5.5 labeled pro-IL-15 protein was injected into MC38 tumor-bearing mice. The protein accumulating within tumor was tracked by IVIS spectrum in vivo imaging system. (b) MC38 tumor bearing mice were i.v. injected with 0.2 μmol pro-IL-15. After 4 days, mice were sacrificed, and tumor, lung, spleen and liver tissue were collected and homogenized. After centrifuge, the supernatant of tissue homogenate and blood were used for immunoprecipitation plus western blotting to detect the sIL-15 and pro-IL-15. (c) MC38 tumor bearing mice (n=5) were treated with 0.2 μmol sIL-15-Fc or pro-IL-15 by i.v. injection on days 8 and 11. (d) A20 tumor bearing mice (n=6) were treated with 0.2 μmol sIL-15-Fc or pro-IL-15 by i.p. injection on days 8 and 11.

FIG. 5. pro-IL-15D1 activate and expand pre-existing CD8⁺ T cells for tumor control. (a),(b) MC38 tumor bearing mice were treated with 0.2 μmol pro-IL-15 by i.p. injection on days 8, 11 and 14. For cell depletion, mice were i.p. injected with 400 μg of anti-NK1.1 Ab (n=6) (a) or 200 μg anti-CD8 Ab (n=5) on day 8, 12 and 16. Tumor growth curve was monitored twice weekly. (c) MC38 tumor bearing mice (n=5) were treated with 0.2 μmol pro-IL-15 by i.p. injection on days 8, 11 and 14. Two days later, tumor tissues were collected. The quantity of T cells in tumor were evaluated by flow cytometry. (d) MC38 tumor bearing mice (n=5) were treated with 0.2 μmol pro-IL-15 by i.p. injection on days 13, 16 and 19. To block T cells migrating into tumor from LN, mice were injected with 25 μg FTY720 on day 13, and next 20 μg FTY720 were given every other day during the experiment. The tumor volume was measured twice weekly. (e) MC38 tumor bearing mice (n=6) were treated with 0.2 μmol pro-IL-15 by i.p. injection on day 10, 14 and 18. For IFN-γ blocking, mice were i.p. injected with 500 μg of anti-IFN-γ Ab (clone R4-6A2) on day 10, 14, 18 and 22. (f),(g) MC38-OVA tumor bearing mice were treated with 0.1 μmol sIL-15-Fc or pro-IL-15 by i.t. injection on days 12 and 15 respectively. Two days later, tumor tissues were harvested and TCF-1⁺ Tim⁻ (e) and PD-1⁺ Tim3⁺ (f) cells in CD8⁺ T cells and were determined by flow cytometry.

FIG. 6. Pro-IL-15D1 synergized with checkpoint blockade to control advanced tumor. (a),(b) sIL-15 treatment increased MDSC infiltration and PDL1 expression within tumor. MC38 tumor bearing mice were treated with 0.2 μmol sIL-15-Fc on day 8 and 11. Three days after first treatment, tumor tissues were collected and single cell suspension was prepared. The MDSC infiltration and PD-L1 expression was analyzed by flow cytometry. (c),(d) MC38 tumor bearing mice (n=5-6) were i.p. treated with 0.3 μmol pro-IL-15 and/or 150 μg anti-PD-L1 Ab on days 12 and 16. The tumor volume was measured twice weekly and recorded as in s(c). Mice survival was recorded as in (d). (e) Naïve mice or Mice with complete tumor regression (n=7) after combination therapy in (c) were re-challenged with 1.5×10⁶ MC38 cells. The tumor growth was monitored twice weekly.

FIG. 7. IL-15 and CD8+ T cells levels positively correlate with better survival in human cancer patients. (a) TCGA data analyze the correlation of IL-15 and CD8⁺ T cells levels within primary or metastasis skin cutaneous melanoma. (b),(c) The correlation of CD8⁺ T cells (b) or IL-15 (c) level within tumor and the survival of primary or metastasis skin cutaneous melanoma patients were analyzed by TIMER.

FIG. 8. sIL-15-Fc have better anti-tumor effect than IL-15-Fc. (a) The activity of indicated proteins were detected by CTLL-2 proliferation assay. (b) A20 tumor bearing mice (n=6) were treated with 0.2 μmol sIL-15-Fc or IL-15-Fc by i.v. injection on day 8 and 11.

FIG. 9. Increased NK but not CD8⁺ or CD4⁺ T cells contribute to sIL-15-Fc treatment induced in vivo toxicity. (a) MC38 tumor-bearing C57BL/6 mice were treated with 0.2 sIL-15-Fc by i.v. injection on day 8 and 11. The lymphocytes expansion in peripheral blood on day 5 after first treatment were determined by FACS. (b) to (d) MC38 tumor-bearing C57BL/6 mice were treated with 0.8 μmol sIL-15-Fc by i.p. injection on day 8 and 11. For cell depletion, mice were i.p. injected with 200 μg of anti-CD8 Ab (b), anti-CD4 Ab (c), or 400 μg of anti-NK1.1 Ab (d) on day 8 and day 11. The survival of the mice was monitored.

FIG. 10. Engineering a tumor-conditional pro-IL-15. (a),(b) A20 tumor bearing mice were treated with 0.2 μmol sIL-15-Fc by i.t. and i.v. injection on day 10 and 13. (b), (c) 3 μg of pro-IL15 was co-cultured with 200 ng pre-activated MMP-14 at 37 degree, After 3 hours, the proteins cleavage was identified by reduced SDS-PAGE (b) and released activity of pro-IL-15 was detected by CTLL2 proliferation assay (c).

FIG. 11. pro-IL-15 induced limited in vivo toxicity. (a),(b) MC38 tumor bearing mice were treated with 0.8 μmol or 1.5 μmol sIL-15 or pro-IL-15 by i.v. injection on day 8 and 11. The mice body weight (a) and survival (b) were monitored. (c) MC38 tumor bearing mice were treated as FIG. 3a. Four days after first treatment, mice were sacrificed and liver tissues were collected for HE staining. (d) C57BL/6 mice were injected i.v. with 30 μg sIL-15-Fc (0.3 μmol) and 30 μg pro-IL-15 (0.2 μmol). Protein concentrations in serum at different time points were measured by ELISA.

FIG. 12. pro-IL-15-FcD1 binds with less peripheral splenocytes. Splenocytes were incubate with indicated protein (hIgG, sIL-15-Fc, pro-IL-15-Fc) for 20 min at 4° C., after twice wash, antibodies to stain B cells, T cells, NK cells, NKT cells were added. The binding of proteins with different cell subsets were detected by secondary antibody goat-anti-hIgG-PE.

FIG. 13. CD4⁺ T cells are not essential for pro-IL-15 mediated anti-tumor activity. (a) MC38 tumor bearing mice were treated with 0.2 μmol pro-IL-15 by i.p. on day 8, 12 and 16. For T cells depletion, 200 μg anti-CD8 Ab and 200 μg anti-CD4 Ab were injected by i.p. on the same day. (b) MC38 tumor bearing mice were treated with 0.2 μmol pro-IL-15 by i.p. on day 8, 12 and 16 and/or 200 μg anti-CD4 Ab were injected by i.p. on the same day.

FIG. 14. Schematic drawing of two homodimeric (model) structures of human pro-sIL15A&B.

FIG. 15. Amino Acid sequences of mRbD(1+2) and mRbD1 and hRbD1 used in constructing pro-sIL15 fusion proteins.

FIG. 16. RbD1-sIL15-Fc shows better anti-tumor effect than Rb-sIL15-Fc: Receptor beta has two domains. D1 reduces blocking but increase potency. C57BL/6J mice were inoculated with 2×10⁵ MC38 cells. After tumor established (10 days), mice were treated with 0.25 μmol Rb-sIL15-mFc (MMP4M) or RbD1-sIL15-mFc (MMP4M) by i.p. injection on day 11, 14 and 18, respectively. RbD1 means D1 of IL15 receptor B.

FIG. 17. Pro-IL15 (RbD1) exhibits less peripheral lymphocytes expansion than IL-15-Fc. C57BL/6 mice were inoculated with 5×10⁵ MC38 cells. After tumor established (about 40 mm³), mice were treated with 0.2 μmol sIL15 with WT or RBD1-IL15-Fc by i.v. injection on day 8 and 11, respectively. Test the lymphocytes expansion in blood on D13 (D5 after treatment) by FACS.

FIG. 18. Different lengths of hRb used to construct pro-sIL15-Fc fusion proteins showed different blocking effect of sIL15 activity. Purified proteins were analyzed on SurePage SDS gel (Genescript) under either non-reducing condition or reducing condition, samples were heated at 95° C. for 5˜10 min before loaded to gel wells. HEKblue-IL15 assay: Seeding 60 μL of 1×10⁶ HEKblue-IL2 cells in assay culture medium: [DMEM-10% FBS(heated)-P/S+HEPES)] into 96 well plate, cultured O/N in 5% CO₂, 37° C. incubator. Next day, perform Serious 1:4 dilution of pro-sIL15-Fc (1102,1172,1188,1103) and control 1100 in assay medium. Add 60 μL of diluted samples and control solutions into HEKblue-IL2 cells seeded plate, cultured O/N in 5% CO₂, 37° C. incubator. Next day, harvest 25 μL of culture supernants from each well, add 80 μL of Quantiblue substrate (Invivogen), incubate 20 min-1 hr @ 37° C. Read plate at 650 nm via ELISA plate reader.

FIG. 19. Analysis of homodimeric pro-sIL15 prodrug B and control fusion proteins. (A), Schematic drawing of homodimeric pro-sIL15B(1118/19) and controls(1085/89 &1199). (B), SDS-PAGE Analysis of purified control and pro-sIL15 prodrug (5.1)+/−mmp14 digestion, showing partial Rb was cut off. C, IL15 activity of purified control and pro-sIL15+/−mmp14 digestion was evaluated on HEKblue-IL2 cells. Rb=(27aa-240aa).

FIG. 20. (Table 1) Production and characterization of hPro-sIL15 fusion proteins using different length of Rb. Insert DNAs were cloned into expression vector via PCR and Gibson Assembly method, and verified by sequencing analysis. Recombinant plasmids were transfected into 293F cells cultured in serum free medium via PEI for 3-5 days. Recombinant proteins in culture supernatants were harvested and purified via protein A affinity column. The blocking effects of sIL15 activity by different lengths of hRb in fusion proteins were tested on HEKblue-IL2 cells (Invivogen), and blocking factor was calculated=EC₅₀ of pro-sIL15-Fc/EC50 of 1100 control (15Ra-Fc− or +/−means no or little expression.

FIG. 21. (Table 2) Example list of long cleavable linker (L2L) with two MMP14 cleavable sites that achieved better cutting showed in vitro. It will not exclude other combinations and orderings. R is reducing gel condition while N is non-reducing gel.

### DEFINITIONS

Unless defined otherwise, all technical and scientific terms used herein have the same meaning as commonly understood by one of ordinary skill in the art to which this invention belongs. The following terms, unless indicated otherwise according to the context wherein the terms are found, are intended to have the following meanings.

When trade names are used herein, the trade name includes the product formulation, the generic drug, and the active pharmaceutical ingredient(s) of the trade name product, unless otherwise indicated by context.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

As used herein, “at least” a specific value is understood to be that value and all values greater than that value.

As used herein, “more than one” is understood as 2, 3, 4, 5, 6, 7, 8, 9, 10, 11, 12, 13, 14, 15, 16, 17, 18, 19, 20, 25, 30, 40, 50, 100, etc., or any value therebetween.

In this specification and the appended claims, the singular forms “a,” “an,” and “the” include plural reference, unless the context clearly dictates otherwise.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein can be modified by the term about.

Unless specifically stated or obvious from context, as used herein, the term “or” is understood to be inclusive.

The term “comprising”, when used to define compositions and methods, is intended to mean that the compositions and methods include the recited elements, but do not exclude other elements. The term “consisting essentially of”, when used to define compositions and methods, shall mean that the compositions and methods include the recited elements and exclude other elements of any essential significance to the compositions and methods. For example, “consisting essentially of” refers to administration of the pharmacologically active agents expressly recited and excludes pharmacologically active agents not expressly recited. The term consisting essentially of does not exclude pharmacologically inactive or inert agents, e.g., pharmaceutically acceptable excipients, carriers or diluents. The term “consisting of”, when used to define compositions and methods, shall mean excluding trace elements of other ingredients and substantial method steps. Embodiments defined by each of these transition terms are within the scope of this invention.

As used herein, the term “agonist” refers to a compound that, in combination with a receptor, can produce a cellular response. An agonist may be a ligand that directly binds to the receptor. Alternatively, an agonist may combine with a receptor indirectly by, for example, (a) forming a complex with another molecule that directly binds to the receptor, or (b) otherwise resulting in the modification of another compound so that the other compound directly binds to the receptor.

As used herein, the term “antagonist” refers to a compound that competes with an agonist or inverse agonist for binding to a receptor, thereby blocking the action of an agonist or inverse agonist on the receptor. However, an antagonist has no effect on constitutive receptor activity.

As used herein, the term “antibody” refers to molecules that are capable of binding an epitope or antigenic determinant. The term is meant to include whole antibodies and antigen-binding fragments thereof. The term encompasses polyclonal, monoclonal, chimeric, Fabs, Fvs, single-chain antibodies and single or multiple immunoglobulin variable chain or CDR domain designs as well as bispecific and multispecific antibodies. Antibodies can be from any animal origin. Preferably, the antibodies are mammalian, e.g., human, murine, rabbit, goat, guinea pig, camel, horse and the like, or other suitable animals. Antibodies may recognize polypeptide or polynucleotide antigens. The term includes active fragments, including for example, an antigen binding fragment of an immunoglobulin, a variable and/or constant region of a heavy chain, a variable and/or constant region of a light chain, a complementarity determining region (cdr), and a framework region. The terms include polyclonal and monoclonal antibody preparations, as well as preparations including hybrid antibodies, altered antibodies, chimeric antibodies, hybrid antibody molecules, F(ab)₂ and F(ab) fragments; Fv molecules (for example, noncovalent heterodimers), dimeric and trimeric antibody fragment constructs; minibodies, humanized antibody molecules, and any functional fragments obtained from such molecules, wherein such fragments retain specific binding.

As used herein, the term “antigen” as used herein is meant any substance that causes the immune system to produce antibodies or specific cell-mediated immune responses against it. A disease associated antigen is any substance that is associated with any disease that causes the immune system to produce antibodies or a specific-cell mediated response against it. An antigen is capable of being recognized by the immune system and/or being capable of inducing a humoral immune response and/or cellular immune response leading to the activation of B- and/or T-lymphocytes. An antigen can have one or more epitopes (B- and/or T-cell epitopes). An antigen will preferably react, typically in a highly selective manner, with its corresponding antibody or TCR and not with the multitude of other antibodies or TCRs which may be evoked by other antigens. Antigens as used herein may also be mixtures of several individual antigens.

As used herein, the term “biologically active” entity, or an entity having “biological activity,” is one having structural, regulatory, or biochemical functions of a naturally occurring molecule or any function related to or associated with a metabolic or physiological process. A biologically active polypeptide or fragment thereof includes one that can participate in a biological process or reaction and/or can produce a desired effect. The biological activity can include an improved desired activity, or a decreased undesirable activity. For example, an entity demonstrates biological activity when it participates in a molecular interaction with another molecule, when it has therapeutic value in alleviating a disease condition, when it has prophylactic value in inducing an immune response, or when it has diagnostic and/or prognostic value in determining the presence of a molecule. A biologically active protein or polypeptide can be naturally-occurring or it can be synthesized from known components, e.g., by recombinant or chemical synthesis and can include heterologous components.

As used herein, the terms “cancer” and “cancerous” refer to or describe the physiological condition in mammals that is typically characterized by unregulated cell growth. Examples of cancer include but are not limited to, carcinoma, lymphoma, sarcoma, blastoma and leukemia. More particular examples of such cancers include squamous cell carcinoma, lung cancer, pancreatic cancer, cervical cancer, bladder cancer, hepatoma, breast cancer, colon carcinoma, and head and neck cancer.

As used herein, the term “cell” refers to any prokaryotic, eukaryotic, primary cell or immortalized cell line, any group of such cells as in, a tissue or an organ. Preferably the cells are of mammalian (e.g., human) origin and can be infected by one or more pathogens.

As used herein, the term “co-administer” refers to the presence of two pharmacological agents in the blood at the same time. The two pharmacological agents can be administered concurrently or sequentially.

As used herein, the term “co-expressed” is intended to mean that two distinct polypeptides are expressed simultaneously in a host cell such that the two polypeptides can interact or bind either in the host cell or in the host cell culture medium and form a complex.

As used herein, the terms “disease” or “disorder” refer to a pathological condition, for example, one that can be identified by symptoms or other identifying factors as diverging from a healthy or a normal state. The term “disease” includes disorders, syndromes, conditions, and injuries. Diseases include, but are not limited to, proliferative, inflammatory, immune, metabolic, infectious, and ischemic diseases.

As used herein, the term “effective amount” of an active agent refers to an amount sufficient to elicit the desired biological response. As will be appreciated by those of ordinary skill in this art, the effective amount of a compound of the invention may vary depending on such factors as the desired biological endpoint, the pharmacokinetics of the compound, the disease being treated, the mode of administration, and the patient.

As used herein, the term “expression of a nucleic acid molecule” refers to the conversion of the information contained in the nucleic acid molecule into a gene product. The gene product can be the direct transcriptional product of a gene (e.g., mRNA, tRNA, rRNA, antisense RNA, ribozyme, structural RNA, or any other type of RNA) or a peptide or polypeptide produced by translation of an mRNA. Gene products also include RNAs that are modified by processes such as capping, polyadenylation, methylation, and editing; and proteins modified by, for example, methylation, acetylation, phosphorylation, ubiquitination, ADP-ribosylation, myristilation, and glycosylation.

As used herein, the term “host cell” refers to an individual cell or a cell culture that can be or has been a recipient of any recombinant vector(s) or isolated polynucleotide(s). A host cell can be a transfected, transformed, transduced or infected cell of any origin, including prokaryotic, eukaryotic, mammalian, avian, insect, plant or bacteria cells, or it can be a cells of any origin that can be used to propagate a nucleic acid described herein. Host cells include progeny of a single host cell, and the progeny may not necessarily be completely identical (in morphology or in total DNA complement) to the original parent cell due to natural, accidental, or deliberate mutation and/or change. A host cell includes cells transfected or infected in vivo or in vitro with a recombinant vector or a polynucleotide of the invention. A host cell that comprises a recombinant vector of the invention may be called a “recombinant host cell.”

Host cells include, without limitation, the cells of mammals, plants, insects, fungi and bacteria. Bacterial cells include, without limitation, the cells of Gram-positive bacteria such as species of the genus Bacillus, Streptomyces and Staphylococcus and cells of Gram-negative bacteria such as cells of the genus Escherichia and Pseudomonas. Fungal cells include, preferably, yeast cells such as Saccharomyces, Pichia pastoris and Hansenula polymorpha. Insect cells include, without limitation, cells of Drosophila and Sf9 cells. Plant cells include, among others, cells from crop plants such as cereals, medicinal or ornamental plants or bulbs. Suitable mammal cells for the present invention include epithelial cell lines (porcine, etc.), osteosarcoma cell lines (human, etc.), neuroblastoma cell lines (human, etc.), epithelial carcinomas (human, etc.), glial cells (murine, etc.), liver cell lines (monkey, etc.). CHO cells (Chinese Hamster Ovary), COS cells, BHK cells, cells HeLa, 911, AT1080, A549, 293 or PER.C6, human ECCs NTERA-2 cells, D3 cells of the line of mESCs, human embryonic stem cells such as HS293 and BGV01, SHEF1, SHEF2 and HS181, cells NIH3T3, 293T, REH and MCF-7 and hMSCs cells.

As used herein, the term “Fc” refers to a molecule or sequence comprising the sequence of a non-antigen-binding fragment of whole antibody, whether in monomeric or multimeric form. The original immunoglobulin source of the native Fc is preferably of human origin and may be any of the immunoglobulins (e.g., IgG1, IgG2). Native Fc's are made up of monomeric polypeptides that may be linked into dimeric or multimeric forms by covalent (i.e., disulfide bonds) and non-covalent association. The number of intermolecular disulfide bonds between monomeric subunits of native Fc molecules ranges from 1 to 4 depending on class (e.g., IgG, IgA, IgE) or subclass (e.g., IgG1, IgG2, IgG3, IgA1, IgGA2).

As used herein, the terms “Fc domain” or “Fc region” is meant to refer to the immunoglobulin heavy chain “fragment crystallizable” region. Generally, an Fc domain is capable of interacting with a second Fc domain to form a dimeric complex. The Fc domain may be capable of binding cell surface receptors called Fc receptors and/or proteins of the complement system or may be modified to reduce or augment these binding activities. The Fc domain may be derived from IgG, IgA, IgD, IgM or IgE antibody isotypes and effect immune activity including opsonization, cell lysis, degranulation of mast cells, basophils, and eosinophils, and other Fc receptor-dependent processes; activation of the complement pathway; and protein stability in vivo.

“Fc domain” encompasses native Fc and Fc variant molecules and sequences as defined herein. As with Fc variants and native Fc's, the term “Fc domain” includes molecules in monomeric or multimeric form, whether digested from whole antibody or produced by recombinant gene expression or by other means.

Fc Fusion proteins have been reported to combine the Fc regions of IgG with the domains of another protein, such as various cytokines and soluble receptors. (e.g., Capon et al. 1989 Nature 337:525-531; Chamow et al. 1996 Trends Biotechnol. 14:52-60; U.S. Pat. Nos. 5,116,964 and 5,541,087).

The use of Fc fusions is known in the art (e.g., U.S. Pat. Nos. 7,754,855; 5,480,981; 5,808,029; WO7/23614; WO98/28427 and references cited therein. Fc fusion proteins can include variant Fc molecules (e.g., as described in U.S. Pat. No. 7,732,570). Fc fusion proteins can be soluble in the plasma or can associate to the cell surface of cells having specific Fc receptors.

As used herein, the term “Fc variant” refers to a molecule or sequence that is modified from a native Fc but still comprises a binding site for the salvage receptor, FcRn. International applications WO 97/34631 (published Sep. 25, 1997) and WO 96/32478 describe exemplary Fc variants, as well as interaction with the salvage receptor, and are hereby incorporated by reference. Thus, the term “Fc variant” comprises a molecule or sequence that is humanized from a non-human native Fc. Furthermore, a native Fc comprises sites that may be removed because they provide structural features or biological activity that are not required for the fusion molecules of the present invention. Thus, in certain embodiments, the term “Fc variant” comprises a molecule or sequence that lacks one or more native Fc sites or residues that affect or are involved in (1) disulfide bond formation, (2) incompatibility with a selected host cell (3) N-terminal heterogeneity upon expression in a selected host cell, (4) glycosylation, (5) interaction with complement, (6) binding to an Fc receptor other than a salvage receptor, or (7) antibody-dependent cellular cytotoxicity (ADCC). Fc variants are described in further detail hereinafter.

As used herein, the term “fusion protein” refers to polypeptides comprising two or more regions from different or heterologous proteins covalently linked (i.e., “fused”) by recombinant, chemical or other suitable method. If desired, the fusion molecule can be fused at one or several sites through a peptide or other linker segment or sequence. For example, one or more peptide linkers may be used to assist in construction of a fusion protein.

As used herein, the term “GC content” refers to the percentage of a nucleic acid sequence comprised of deoxyguanosine (G) and/or deoxycytidine (C) deoxyribonucleosides, or guanosine (G) and/or cytidine (C) ribonucleoside residues.

As used herein, the term “high dosage” is meant at least 5% (e.g., at least 10%, 20%, 50%, 100%, 200%, or even 300%) more than the highest standard recommended dosage of a particular compound for treatment of any human disease or condition.

As used herein, the term “immune response” refers to a process whereby immune cells are stimulated and/or recruited from the blood to lymphoid as well as non-lymphoid tissues via a multifactorial process that involves distinct adhesive and/or activation steps. Activation conditions cause the release of cytokines, growth factors, chemokines and other factors, upregulate expression of adhesion and other activation molecules on the immune cells, promote adhesion, morphological changes, and/or extravasation concurrent with chemotaxis through the tissues, increase cell proliferation and cytotoxic activity, stimulate antigen presentation and provide other phenotypic changes including generation of memory cell types. Immune response is also meant to refer to the activity of immune cells to suppress or regulate inflammatory or cytotoxic activity of other immune cells. Immune response refers to the activity of immune cells in vivo or in vitro.

The terms “identical” or percent “identity,” in the context of two or more nucleic acids or polypeptide sequences, refer to two or more sequences or subsequences that are the same or have a specified percentage of amino acid residues or nucleotides that are the same (i.e., about 70% identity, preferably 75%, 80%, 85%, 90%, 91%, 92%, 93%, 94%, 95%, 96%, 97%, 98%, 99%, or higher identity over a specified region (e.g., of a IL15 or IL15Rα sequence), when compared and aligned for maximum correspondence over a comparison window or designated region) as measured using a BLAST or BLAST 2.0 sequence comparison algorithms with default parameters described below, or by manual alignment and visual inspection. Such sequences are then said to be “substantially identical.” This definition also refers to, or can be applied to, the compliment of a test sequence. The definition also includes sequences that have deletions and/or additions, as well as those that have substitutions. As described below, the preferred algorithms can account for gaps and the like. Preferably, identity exists over a region that is at least about 25, 50, 75, 100, 150, 200 amino acids or nucleotides in length, and oftentimes over a region that is 225, 250, 300, 350, 400, 450, 500 amino acids or nucleotides in length or over the full-length of an amino acid or nucleic acid sequences.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Preferably, default program parameters can be used, or alternative parameters can be designated. The sequence comparison algorithm then calculates the percent sequence identities for the test sequences relative to the reference sequence, based on the program parameters.

A preferred example of algorithm that is suitable for determining percent sequence identity and sequence similarity are the BLAST algorithms, which are described in Altschul et al. 1977 Nuc. Acids Res. 25:3389-3402 and Altschul et al. 1990 J. Mol. Biol. 215:403-410, respectively. BLAST software is publicly available through the National Center for Biotechnology Information on the worldwide web at ncbi.nlm.nih.gov/. Both default parameters or other non-default parameters can be used. The BLASTN program (for nucleotide sequences) uses as defaults a wordlength (W) of 11, an expectation (E) of 10, M=5, N=−4 and a comparison of both strands. For amino acid sequences, the BLASTP program uses as defaults a wordlength of 3, and expectation (E) of 10, and the BLOSUM62 scoring matrix (see Henikoff & Henikoff, Proc. Natl. Acad. Sci. USA 89:10915 (1989)) alignments (B) of 50, expectation (E) of 10, M=5, N=−4, and a comparison of both strands.

As used herein, the term “inhibit” refers to any measurable reduction of biological activity. Thus, as used herein, “inhibit” or “inhibition” may be referred to as a percentage of a normal level of activity.

As used herein, the term “interleukin 15” or “IL15” refers to a polypeptide that has at least 90%, 91%, 92%, 93%, 94%, 95%, 96%, 97%, 98% or 99% sequence identity to a native mammalian IL15 amino acid sequence that is biologically active, meaning the mutated protein (“mutein”) has functionality similar (75% or greater) to that of a native IL15 protein in at least one functional assay. Functionally, IL15 is a cytokine that regulates T cell and natural killer cell activation and proliferation.

IL15 and IL2 share many biological activities, including binding to CD122, the IL2β/IL15β receptor subunit. The number of CD8+ memory cells is controlled by a balance between this IL15 and IL2. IL15 induces the activation of JAK kinases, as well as the phosphorylation and activation of transcription activators STAT3, STATS, and STAT6. IL15 also increases the expression of apoptosis inhibitor BCL2L1/BCL-x (L), possibly through the transcription activation activity of STAT6, and thus prevents apoptosis. Two alternatively spliced transcript variants of the IL15 gene encoding the same mature protein have been reported.

Exemplified functional assays of an IL15 polypeptide include proliferation of T-cells (e.g., Montes et al. 2005 Clin Exp Immunol 142:292), and activation of NK cells, macrophages and neutrophils. Methods for isolation of particular immune cell subpopulations and detection of proliferation (i.e., ³H-thymidine incorporation) are well known in the art. Cell-mediated cellular cytotoxicity assays can be used to measure NK cell, macrophage and neutrophil activation. Cell-mediated cellular cytotoxicity assays, including release of isotopes (⁵¹Cr), dyes (e.g., tetrazolium, neutral red) or enzymes, are also well known in the art, with commercially available kits (Oxford Biomedical Research, Oxford, M; Cambrex, Walkersville, Md.; Invitrogen, Carlsbad, Calif.). IL15 has also been shown to inhibit Fas mediated apoptosis (e.g., Demirci et al. 2004 Cell Mol Immunol 1:123). Apoptosis assays, including for example, TUNEL assays and annexin V assays, are well known in the art with commercially available kits (R&D Systems, Minneapolis, Minn.). (e.g., Coliga et al. 1991-2006 Current Methods in Immunology John Wiley & Sons.)

As used herein, the term “interleukin 15 receptor alpha” or “IL15Rα” refers to an interleukin 15 receptor alpha amino acid sequences from a mammalian species. Those of skill in the art will appreciate that interleukin-15 receptor alpha nucleic acid and amino acid sequences are publicly available in gene databases, for example, GenBank through the National Center for Biotechnological Information on the worldwide web at ncbi.nlm.nih.gov. Exemplified native mammalian IL-15 receptor alpha nucleic acid or amino acid sequences can be from, for example, human, primate, canine, feline, porcine, equine, bovine, ovine, rodentia, murine, rat, hamster, guinea pig, etc. Accession numbers for exemplary native mammalian IL-15 nucleic acid sequences include NM_172200.1 (human isoform 2); and NM_002189.2 (human isoform 1 precursor). Accession numbers for exemplary native mammalian IL-15 amino acid sequences include NP_751950.1 (human isoform 2); and NP_002180.1 (human isoform 1 precursor).

As used herein, “interleukin 15 receptor alpha” or “IL15Rα” may also refer to a polypeptide that has at least 90%, 91%, 92%, 93%, 94%, 95%, 96%, 97%, 98% or 99% sequence identity to a native mammalian IL15Rα amino acid sequence that is biologically active, having functionality similar (75% or greater) to that of a native IL15Rα protein in at least one functional assay. IL15Rα is a cytokine receptor that specifically binds IL15 with high affinity. One functional assay is specific binding to a native IL15 protein.

As used herein, the terms an “isolated” molecule (such as a polypeptide or polynucleotide) is one that has been manipulated to exist in a higher concentration than in nature or has been removed from its native environment. For example, a subject antibody is isolated, purified, substantially isolated, or substantially purified when at least 10%, or 20%, or 40%, or 50%, or 70%, or 90% of non-subject-antibody materials with which it is associated in nature have been removed. For example, a polynucleotide or a polypeptide naturally present in a living animal is not “isolated,” but the same polynucleotide or polypeptide separated from the coexisting materials of its natural state is “isolated.” Further, recombinant DNA molecules contained in a vector are considered isolated for the purposes of the present invention. Isolated RNA molecules include in vivo or in vitro RNA replication products of DNA and RNA molecules. Isolated nucleic acid molecules further include synthetically produced molecules. Additionally, vector molecules contained in recombinant host cells are also isolated. Thus, not all “isolated” molecules need be “purified.”

As used herein, the terms “linker” or “linking segment” refer to a molecule or group that connects two other molecules or groups. A peptide linker may allow the connected molecules or groups to acquire a functional configuration. The linker peptide preferably comprises at least two amino acids, at least three amino acids, at least five amino acids, at least ten amino acids, at least 15 amino acids, at least 20 amino acids, at least 30 amino acids, at least 40 amino acids, at least 50 amino acids, at least 60 amino acids, at least 70 amino acids, at least 80 amino acids, at least 90 amino acids or approximately 100 amino acids.

Components of a fusion protein, such as cytokines or other bioactive molecules and any peptide linkers, can be organized in nearly any fashion provided that the fusion protein has the function for which it was intended. In particular, each component of a fusion protein can be spaced from another component by at least one suitable peptide linker segment or sequence if desired. Additionally, the fusion protein may include tags, e.g., to facilitate modification, identification and/or purification of the fusion protein. More specific fusion proteins are in the examples described below.

As used herein, the term “low dosage” refers to at least 5% less (e.g., at least 10%, 20%, 50%, 80%, 90%, or even 95%) than the lowest standard recommended dosage of a particular compound formulated for a given route of administration for treatment of any human disease or condition. For example, a low dosage of an agent that is formulated for administration by inhalation will differ from a low dosage of the same agent formulated for oral administration.

As used herein, the term “medium” or “media” includes any culture medium, solution, solid, semi-solid, or rigid support that may support or contain any host cell, including bacterial host cells, yeast host cells, insect host cells, plant host cells, eukaryotic host cells, mammalian host cells, CHO cells, prokaryotic host cells, E. coli, or Pseudomonas host cells, and cell contents. Thus, the term may encompass medium in which the host cell has been grown, e.g., medium into which a polypeptide has been secreted, including medium either before or after a proliferation step. The term also may encompass buffers or reagents that contain host cell lysates, such as in the case where a polypeptide is produced intracellularly and the host cells are lysed or disrupted to release the polypeptide.

As used herein, the term “modulate” refers to the production, either directly or indirectly, of an increase or a decrease, a stimulation, inhibition, interference, or blockage in a measured activity when compared to a suitable control. A “modulator” of a polypeptide or polynucleotide refers to a substance that affects, for example, increases, decreases, stimulates, inhibits, interferes with, or blocks a measured activity of the polypeptide or polynucleotide, when compared to a suitable control. For example, a “modulator” may bind to and/or activate or inhibit the target with measurable affinity, or directly or indirectly affect the normal regulation of a receptor activity.

The term “operably linked” refers to a functional linkage between a first nucleic acid sequence and a second nucleic acid sequence, such that the first and second nucleic acid sequences are transcribed into a single nucleic acid sequence. Operably linked nucleic acid sequences need not be physically adjacent to each other. The term “operably linked” also refers to a functional linkage between a nucleic acid expression control sequence (such as a promoter, or array of transcription factor binding sites) and a transcribable nucleic acid sequence, wherein the expression control sequence directs transcription of the nucleic acid corresponding to the transcribable sequence.

As used herein, the term “pharmaceutically acceptable” excipient, carrier, or diluent refers to a pharmaceutically acceptable material, composition or vehicle, such as a liquid or solid filler, diluent, excipient, solvent or encapsulating material, involved in carrying or transporting the subject pharmaceutical agent from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations. Wetting agents, emulsifiers and lubricants, such as sodium lauryl sulfate, magnesium stearate, and polyethylene oxide-polypropylene oxide copolymer as well as coloring agents, release agents, coating agents, sweetening, flavoring and perfuming agents, preservatives and antioxidants can also be present in the compositions.

As used herein, the terms “polynucleotide,” “nucleic acid molecule,” “nucleotide,” “oligonucleotide,” and “nucleic acid” are used interchangeably herein to refer to polymeric forms of nucleotides, including ribonucleotides as well as deoxyribonucleotides, of any length. They can include both double-, single-stranded or triple helical sequences and include, but are not limited to, cDNA from viral, prokaryotic, and eukaryotic sources; mRNA; genomic DNA sequences from viral (e.g., DNA viruses and retroviruses) or prokaryotic sources; RNAi; cRNA; antisense molecules; recombinant polynucleotides; ribozymes; and synthetic DNA sequences. The term also captures sequences that include any of the known base analogs of DNA and RNA. Nucleotides can be referred to by their commonly accepted single-letter codes.

Polynucleotides are not limited to polynucleotides as they appear in nature, and also include polynucleotides where unnatural nucleotide analogues and inter-nucleotide bonds appear. A nucleic acid molecule may comprise modified nucleic acid molecules (e.g., modified bases, sugars, and/or internucleotide linkers). Non-limitative examples of this type of unnatural structures include polynucleotides wherein the sugar is different from ribose, polynucleotides wherein the phosphodiester bonds 3′-5′ and 2′-5′ appear, polynucleotides wherein inverted bonds (3′-3′ and 5′-5′) appear and branched structures. Also, the polynucleotides of the invention include unnatural inter-nucleotide bonds such as peptide nucleic acids (PNA), locked nucleic acids (LNA), C1-C4 alkylphosphonate bonds of the methylphosphonate, phosphoramidate, C1-C6 alkylphosphotriester, phosphorothioate and phosphorodithioate type. In any case, the polynucleotides of the invention maintain the capacity to hybridize with target nucleic acids in a similar way to natural polynucleotides.

Unless otherwise indicated or obvious from context, a particular nucleic acid sequence also implicitly encompasses conservatively modified variants thereof (e.g., degenerate codon substitutions) and complementary sequences, as well as the sequence explicitly indicated. Degenerate codon substitutions can be achieved by generating sequences in which the third position of one or more selected (or all) codons is substituted with mixed-base and/or deoxyinosine residues. (Batzer et al. 1991 Nucleic Acid Res. 19:5081; Ohtsuka et al. 1985 J. Biol. Chem. 260:2605-2608; Rossolini et al. 1994 Mol. Cell. Probes 8:91-98.)

As used herein, the terms “prevent”, “preventing”, or “prevention” refer to a method for precluding, delaying, averting, or stopping the onset, incidence, severity, or recurrence of a disease or condition. For example, a method is considered to be a prevention if there is a reduction or delay in onset, incidence, severity, or recurrence of a disease or condition or one or more symptoms thereof in a subject susceptible to the disease or condition as compared to a subject not receiving the method. The disclosed method is also considered to be a prevention if there is a reduction or delay in onset, incidence, severity, or recurrence of one or more symptoms of a disease or condition in a subject susceptible to the disease or condition after receiving the method as compared to the subject's progression prior to receiving treatment. The reduction or delay in onset, incidence, severity, or recurrence of osteoporosis can be about a 5, 10, 20, 30, 40, 50, 60, 70, 80, 90, 100%, or any amount of reduction in between.

Prevention and the like do not mean preventing a subject from ever getting the specific disease or disorder. Prevention may require the administration of multiple doses. Prevention can include the prevention of a recurrence of a disease in a subject for whom all disease symptoms were eliminated, or prevention of recurrence in a relapsing-remitting disease.

As used herein, the term “promoter” refers to a DNA regulatory region capable of binding RNA polymerase in a mammalian cell and initiating transcription of a downstream (3′ direction) coding sequence operably linked thereto. A promoter sequence includes the minimum number of bases or elements necessary to initiate transcription of a gene of interest at levels detectable above background. Within the promoter sequence may be a transcription initiation site, as well as protein binding domains (consensus sequences) responsible for the binding of RNA polymerase. Eukaryotic promoters will often, but not always, contain “TATA” boxes and “CAT” boxes. Promoters include those that are naturally contiguous to a nucleic acid molecule and those that are not naturally contiguous to a nucleic acid molecule. Additionally, the term “promoter” includes inducible promoters, conditionally active promoters such as a cre-lox promoter, constitutive promoters, and tissue specific promoters.

As used herein, the terms “protein” and “polypeptide” are used interchangeably to refer to a polymer of amino acid residues, and are not limited to a minimum length. Thus, peptides, oligopeptides, dimers, multimers, and the like, are included within the definition. Both full-length proteins and fragments thereof are encompassed by the definition. The terms also include post-expression modifications of the polypeptide, for example, glycosylation, acetylation, phosphorylation, and the like. Furthermore, a polypeptide may refer to a protein which includes modifications, such as deletions, additions, and substitutions (generally conservative in nature), to the native sequence, as long as the protein maintains the desired activity. These modifications may be deliberate or may be accidental. Amino acids can be referred to herein by either their commonly known three letter symbols or by the one-letter symbols recommended by the IUPAC-IUB Biochemical Nomenclature Commission.

As used herein, the term “purified” refers to a protein that may be substantially or essentially free of components that normally accompany or interact with the protein as found in its naturally occurring environment, i.e. a native cell, or host cell in the case of a recombinantly produced protein. A protein that may be substantially free of cellular material includes preparations of protein having less than about 30%, less than about 20%, less than about 15%, less than about 10%, less than about 5%, less than about 4%, less than about 3%, less than about 2%, or less than about 1% (by dry weight) of contaminating protein(s). When a protein or variant thereof is recombinantly produced by the host cells, the protein may be present at about 30%, at about 20%, about 15%, about 10%, about 5%, about 4%, about 3%, about 2%, or about 1% or less of the dry weight of the cells. When a protein or variant thereof is recombinantly produced by the host cells, the protein may be present in the culture medium at about 5 g/L, about 4 g/L, about 3 g/L, about 2 g/L, about 1 g/L, about 750 mg/L, about 500 mg/L, about 250 mg/L, about 100 mg/L, about 50 mg/L, about 10 mg/L, or about 1 mg/L or less of the dry weight of the cells. Thus, a “substantially purified” protein may have a purity level of at least at least about 80%, specifically, a purity level of at least about 85%, and more specifically, a purity level of at least about 90%, a purity level of at least about 95%, a purity level of at least about 99% or greater as determined by appropriate methods such as SDS/PAGE analysis, RP-HPLC, SEC, and capillary electrophoresis.

Proteins and prodrugs of the present invention are, subsequent to their preparation, preferably isolated and/or purified to obtain a composition containing an amount by weight equal to or greater than 80% (“substantially pure”), which is then used or formulated as described herein. In certain embodiments, the compounds of the present invention are more than 95% pure.

As used herein, the term “receptor” refers to proteins, including glycoproteins or fragments thereof, capable of interacting with another molecule, called the ligand. The ligand may belong to any class of biochemical or chemical compounds. The ligand is usually an extracellular molecule which, upon binding to the receptor, usually initiates a cellular response, such as initiation of a signal transduction pathway. The receptor need not necessarily be a membrane-bound protein.

As used herein, the term “recombinant,” with respect to a nucleic acid molecule, means a polynucleotide of genomic, cDNA, viral, semisynthetic, and/or synthetic origin which, by virtue of its origin or manipulation, is not associated with all or a portion of the polynucleotide with which it is associated in nature. The term “recombinant”, as used with respect to a protein or polypeptide, means a polypeptide produced by expression of a recombinant polynucleotide. The term “recombinant” as used with respect to a host cell means a host cell into which a recombinant polynucleotide has been introduced.

As used herein, the term “sample” refers to a sample from a human, animal, or to a research sample, e.g., a cell, tissue, organ, fluid, gas, aerosol, slurry, colloid, or coagulated material. The “sample” may be tested in vivo, e.g., without removal from the human or animal, or it may be tested in vitro. The sample may be tested after processing, e.g., by histological methods. “Sample” also refers, e.g., to a cell comprising a fluid or tissue sample or a cell separated from a fluid or tissue sample. “Sample” may also refer to a cell, tissue, organ, or fluid that is freshly taken from a human or animal, or to a cell, tissue, organ, or fluid that is processed or stored.

As used herein, the term “soluble” refers to a fusion molecule, particularly a fusion protein, that is not readily sedimented under low G-force centrifugation (e.g., less than about 30,000 revolutions per minute in a standard centrifuge) from an aqueous buffer, e.g., cell media. A fusion molecule is soluble if it remains in aqueous solution at a temperature greater than about 5-37° C. and at or near neutral pH in the presence of low or no concentration of an anionic or non-ionic detergent. Under these conditions, a soluble protein will often have a low sedimentation value, e.g., less than about 10 to 50 Svedberg units.

Aqueous solutions referenced herein typically have a buffering compound to establish pH, typically within a pH range of about 5-9, and an ionic strength range between about 2 mM and 500 mM. Sometimes a protease inhibitor or mild non-ionic detergent is added. Additionally, a carrier protein may be added if desired (e.g., bovine serum albumin). Exemplary aqueous buffers include standard phosphate buffered saline, tris-buffered saline, or other well-known buffers and cell media formulations.

As used herein, the term “soluble IL15 Receptor alpha” refers to forms of IL15 Receptor alpha lacking the transmembrane anchor portion of the receptor and thus able to be secreted out of the cell without being anchored to the plasma membrane.

As used herein, the terms “stimulate” or “stimulating” refer to increase, to amplify, to augment, to boost a physiological activity, e.g., an immune response. Stimulation can be a positive alteration. For example, an increase can be by 5%, 10%, 25%, 50%, 75%, or even 90-100%. Other exemplary increases include 2-fold, 5-fold, 10-fold, 20-fold, 40-fold, or even 100-fold.

As used herein, the terms “subject” and “patient” are used interchangeably herein to refer to a living animal (human or non-human). The subject may be a mammal. The terms “mammal” or “mammalian” refer to any animal within the taxonomic classification mammalia. A mammal may be a human or a non-human mammal, for example, dogs, cats, pigs, cows, sheep, goats, horses, rats, and mice. The term “subject” does not preclude individuals that are entirely normal with respect to a disease or condition, or normal in all respects.

As used herein, the terms “suppress” or “suppressing” refer to decrease, to attenuate, to diminish, to arrest, or to stabilize a physiological activity, e.g., an immune response. Suppression can be a negative alteration. For example, a decrease can be by 5%, 10%, 25%, 50%, 75%, or even 90-100%. Exemplary decreases include 2-fold, 5-fold, 10-fold, 20-fold, 40-fold, or even 100-fold.

As used herein, the term “therapeutically effective amount” refers to the dose of a therapeutic agent or agents sufficient to achieve the intended therapeutic effect with minimal or no undesirable side effects. A therapeutically effective amount can be readily determined by a skilled physician, e.g., by first administering a low dose of the pharmacological agent(s) and then incrementally increasing the dose until the desired therapeutic effect is achieved with minimal or no undesirable side effects.

As used herein, the term “transfected” means possessing introduced DNA or RNA, with or without the use of any accompanying facilitating agents such as lipofectamine. Methods for transfection that are known in the art include, for example, calcium phosphate transfection, DEAE dextran transfection, protoplast fusion, electroporation, and lipofection.

As used herein, the terms “treatment” or “treating” a disease or disorder refers to a method of reducing, delaying or ameliorating such a condition, or one or more symptoms of such disease or condition, before or after it has occurred. Treatment may be directed at one or more effects or symptoms of a disease and/or the underlying pathology. The treatment can be any reduction and can be, but is not limited to, the complete ablation of the disease or the symptoms of the disease. As compared with an equivalent untreated control, such reduction or degree of prevention is at least 5%, 10%, 20%, 40%, 50%, 60%, 80%, 90%, 95%, or 100% as measured by any standard technique.

As used herein, the term “tumor” refers to any malignant or neoplastic cell.

As used herein, the term “vector” refers to a nucleic acid molecule that is able to transmit genetic material to a host cell or organism. A vector may be composed of either DNA or RNA. A vector carries its own origin of replication, one or more unique recognition sites for restriction endonucleases which can be used for the insertion of foreign DNA, and usually selectable markers such as genes coding for antibiotic resistance, and often recognition sequences (e.g., promoter) for the expression of the inserted DNA. Common vectors include plasmid vectors and phage vectors.

Any compositions or methods disclosed herein can be combined with one or more of any of the other compositions and methods provided herein.

### DETAILED DESCRIPTION OF THE INVENTION

The invention provides novel fusion proteins and therapeutic uses thereof. More particularly, the invention provides novel fusion proteins of IL15 and prodrugs thereof, compositions and methods of preparation thereof, which are useful in treating various diseases and disorders, e.g., hyperplasia, solid tumor or hematopoietic malignancy with reduced off-target toxicities and side effects during treatment. In particular and by way of example, the present employs a shorter domain of IL15 receptor β, as opposed to the whole extracellular IL15 receptor β (27aa-240aa), to block super IL15 (IL15-Rα), where the blockage is removed and sIL15 activity is recovered inside a tumor microenvironment. This approach led to an improved antitumor activity without increasing its toxicity profile. As disclosed herein, various short forms of hIL15 receptor β were used to construct pro-sIL15 fusion proteins, including domain 1 (D1) corresponding to 27aa-125aa, along with various different cleavable flexible linkers (L2 or L2L).

Various formats of pro-sIL15 fusion protein or fusion protein complex are disclosed herein. For example, disclosed herein is a pro-sIL15 A as tumor-conditional targeting and activation. The extracellular domain of IL15RβD1 is fused to the N-terminal of IL15-IL15Rα-Fc by an MMP-14 cleavable peptide linker. It was found that this targeted pro-sIL15 overcame off-tumor expansion of NK cell caused systemic toxicity and efficiently inhibited tumor growth. The anti-tumor effect of pro-sIL15 depend on CD8⁺ T cell. Pro-IL15 increased the stem-like (TCF1⁺Tim-3⁻) CD8⁺ T cells and decreased the portion of CD8⁺ T cells with terminal phenotype (PD1⁺Tim3^{hi}). The combination with anti-PD-L1 treatment significantly increase the therapeutic effect of pro-IL-15 and can clear most tumor completely.

In one aspect, the invention generally relates to a fusion protein or fusion protein complex (A), being a homodimeric complex and comprising:
a first structural unit: a whole or a subunit domain of the interleukin 15 (IL15) receptor-α;
a second structural unit: an active IL15;
a third structural unit located at the C-terminus of the fusion protein: an antibody Fc fragment;
a fourth structural unit located at the N-terminus of the fusion protein: a whole or a subunit domain of the IL15 receptor β linked to the first, second or third structure unit via a short cleavable linker (L2) or long cleavable linker (L2L); and
one or more connecting segments or ligation fragments as flexible non-cleavable linkers (L1) for connecting the different units.

In another aspect, the invention generally relates to a fusion protein or fusion protein complex (B), being a homodimeric complex and comprising:
a first fragment (Fragment No. 1), comprising:
   a first structural unit: a whole or a subunit domain of the interleukin 15 (IL15) receptor-α, and
   a fifth structural unit: a constant region of light chain (CL); and
a second fragment (Fragment No. 2), comprising:
   a second structural unit: an active IL15,
   a third structural unit located at the C-terminus of the fusion protein: an antibody Fc fragment,
   a fourth structural unit located at the N-terminus of the fusion protein: a whole or a subunit domain of the IL15 receptor β; and linked with either short cleavable linker (L2) or long cleavable linker (L2L),
   a sixth structural unit: a constant region of heavy chain (CH), and
   one or more connecting segments or ligation fragments as flexible non-cleavable linkers (L1) for connecting the different units,
      wherein two Fragment No. 1 and two Fragment No. 2 are joined together to form a complex via disulfide bonds between CH and CL and between two Fc's.

In certain embodiments, the active IL15 is a human IL15.

In certain embodiments the antibody Fc fragment comprises a human Fc fragment having the amino acid sequence set forth in SEQ ID No 2.

In certain embodiments of the fusion protein, the human Fc fragment comprises a mutant human IgG1-Fc having the amino acid sequence set forth in SEQ ID No. 3.

In certain embodiments, the first structural unit is a whole interleukin 15 (IL15) receptor-α.

In certain embodiments, the first structural unit is a subunit domain of interleukin 15 (IL15) receptor-α.

In certain embodiments, the first structural unit is the sushi domain of a subunit of human IL15R, with the amino acid sequence set forth in SEQ ID No. 4.

In certain embodiments, the fourth structural unit is the whole extra cellular domain(27aa˜240aa) of human IL15Rb, with the amino acid sequence set forth in SEQ ID No. 5.

In certain embodiments, the fourth structural unit is a subunit domain of the IL15 receptor β having shorter length than extra cellular 27aa-240aa of IL15 receptor β.

In certain embodiments, the subunit domain of the IL15 receptor β is selected from a shortened IL15 receptor β represented by 27aa-125aa, 27aa-128aa, 27aa-137aa, 27˜234aa, 32aa-1378aa, 32aa-234aa, 32aa-240aa, 83aa˜214aa, 83˜234aa, or 83-240aa, set forth in SEQ ID No 6˜15.

In certain embodiments, the subunit domain of the IL15 receptor β is a shortened IL15 receptor β represented by 27-125aa.

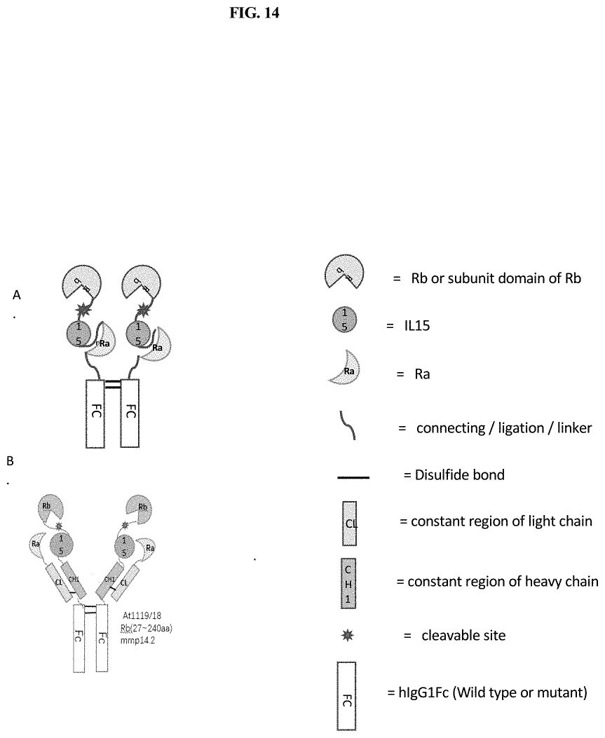

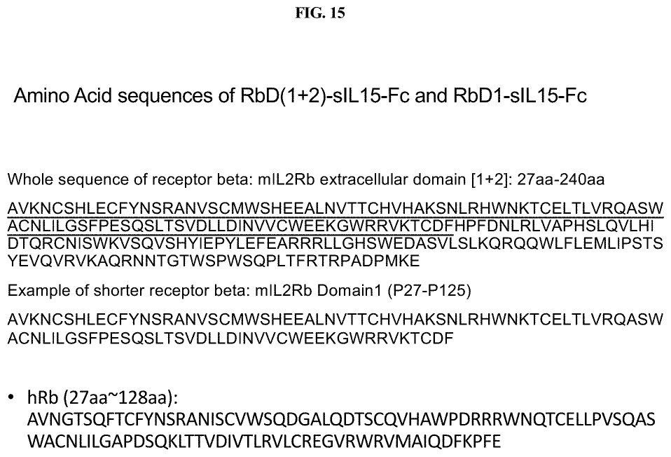

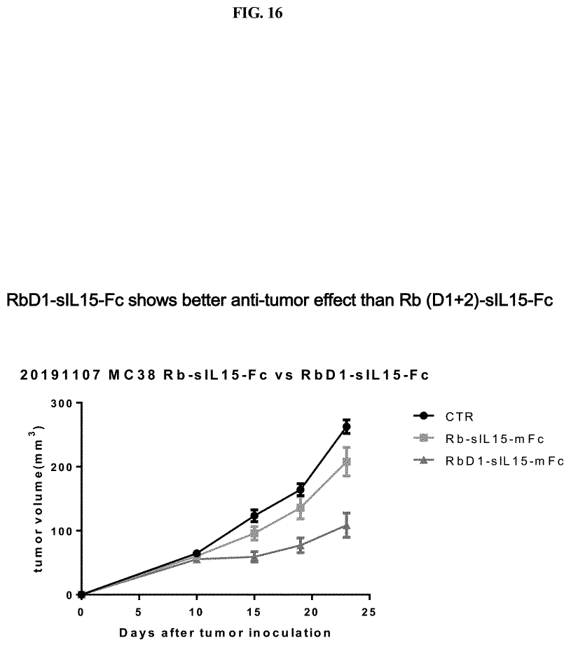

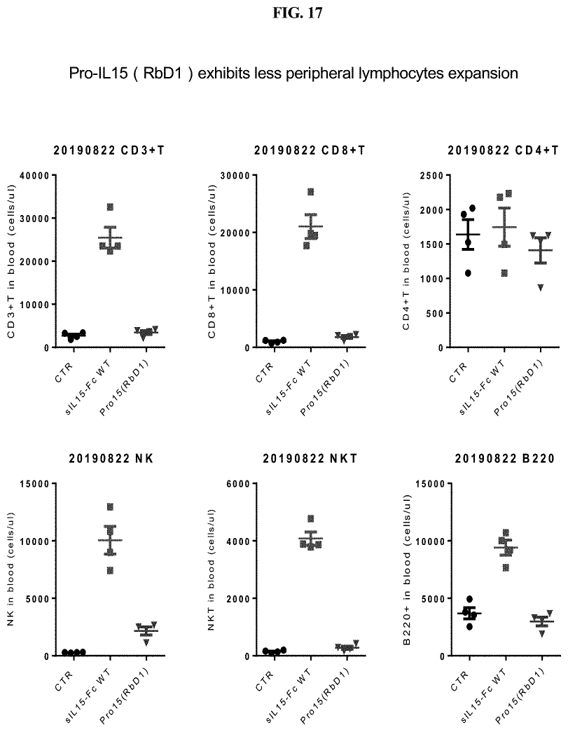

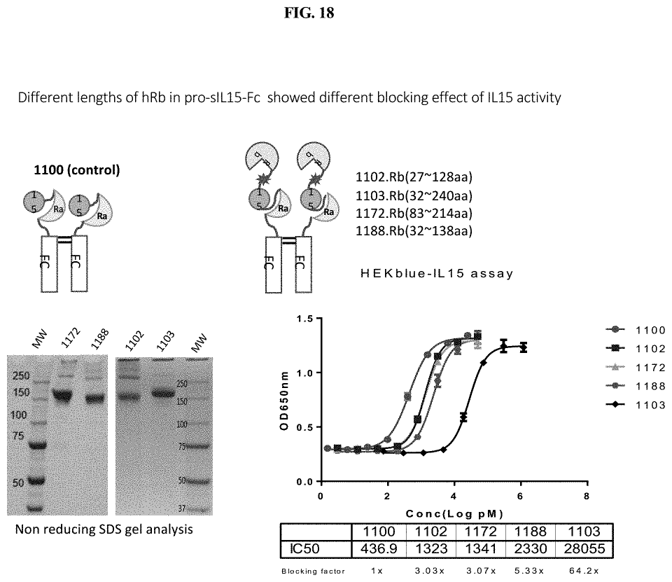

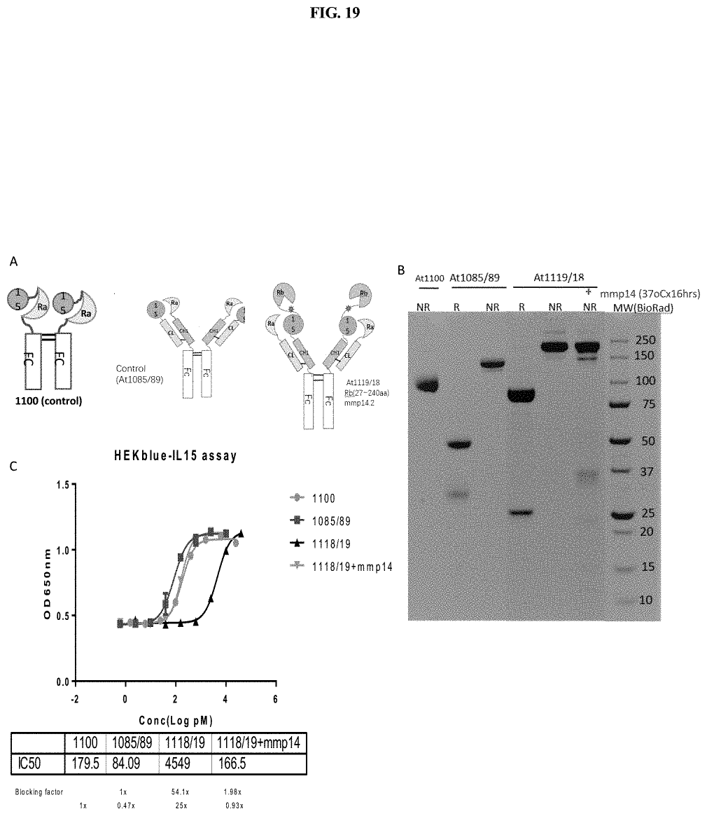

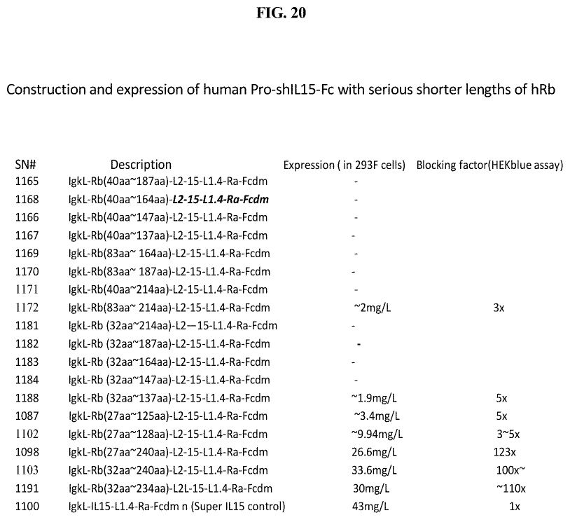

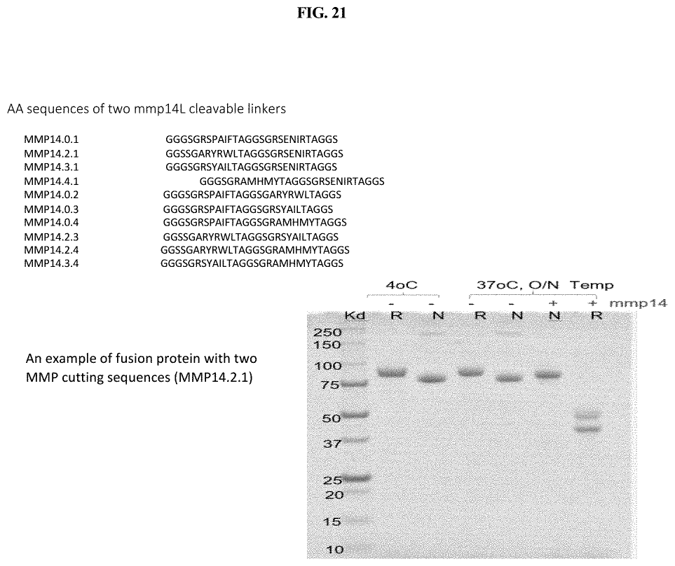

In certain embodiments, the fusion protein or fusion protein complex has a construct as shown in FIG. 14A.

In certain embodiments, the fusion protein or fusion protein complex has a construct as shown in FIG. 14B.

In certain embodiments, each of the one or more connecting segments or ligation fragments is selected from a cleavable flexible linker (L2 or L2L) or a noncleavable flexible linker (L1). In certain embodiments, the short cleavable flexible linker (L2) and the long cleavable flexible linker (L2L) are capable of being recognized and hydrolyzed by a proteolytic enzyme specifically expressed in a tumor microenvironment.

As used herein, the term “short cleavable linker” or linker “L2” refers to cleavable linkers having a length in the range of about 10 to about 20 with one cleavable site. Non-limiting examples of short cleavable linkers are listed in SEQ. ID. 17. As used herein, the term “long cleavable linker” or linker “L2L” refers to cleavable linkers having a length in the range of about 20 to about 40. In certain embodiments, a long cleavable linker (L2L) comprises two cleavable sites. Non-limiting examples of non-cleavable linkers are listed in SEQ. ID. 16. Non-limiting examples of long cleavable linkers are listed in SEQ. ID. 18.

In certain embodiments, one of the connecting segments or linking fragments comprises an amino acid sequence that is a multiple of GGGS with or without protease sensitive sequences.

In certain embodiments, one or more connecting segments or linkers is capable of being recognized and hydrolyzed by a proteolytic enzyme specifically expressed in a tumor microenvironment.

In certain embodiments, the proteolytic enzyme specifically expressed in the tumor microenvironment is a matrix metalloproteinase.

In certain embodiments, the matrix metalloproteinase is matrix metalloproteinase 9 (MMP9).

In certain embodiments, the matrix metalloproteinase is matrix metalloproteinase 14 (MMP14).

In certain embodiments, a connecting segment or linkers comprises the amino acid sequence set forth in SEQ ID No. 8.

In certain embodiments, a connecting segment or linkers comprises the amino acid sequence set forth in SEQ ID No. 9.

In certain embodiments, a connecting segment or linkers comprises an amino acid sequence set forth in SEQ ID Nos. 10-23.

In certain embodiments, hydrolysis of the connecting segment or linkers separates the subunit domain of the IL15 receptor β from the rest of the fusion protein.

In certain embodiments, a second connecting segment or linkers is capable of being recognized and hydrolyzed by a proteolytic enzyme specifically expressed in a tumor microenvironment.

In certain embodiments, the proteolytic enzyme specifically expressed in the tumor microenvironment is a matrix metalloproteinase.

In yet another aspect, the invention generally relates to a homodimeric protein or fusion protein complex, comprising a fusion protein disclosed herein.

In yet another aspect, the invention generally relates to a prodrug comprising a fusion protein or fusion protein complex disclosed herein.

In certain embodiments, the homodimeric protein or fusion protein complex, or prodrug thereof, is hydrolyzed by a proteolytic enzyme specifically expressed in a tumor microenvironment.

In yet another aspect, the invention generally relates to a substantially purified fusion protein or fusion protein complex, or prodrug thereof, disclosed herein.

In yet another aspect, the invention generally relates to a polynucleotide encoding a fusion protein or fusion protein complex, or prodrug thereof, disclosed herein.

In yet another aspect, the invention generally relates to an expression vector comprising a polynucleotide disclosed herein.

In yet another aspect, the invention generally relates to a pharmaceutical composition comprising a fusion protein or fusion protein complex, or prodrug thereof, disclosed herein.

In yet another aspect, the invention generally relates to a method for treating a disease or condition. The method comprises administering to a patient in need thereof a therapeutically effective amount of a fusion protein or fusion protein complex, or prodrug thereof, or a pharmaceutical composition disclosed herein, wherein the disease or condition is selected from hyperplasia, solid tumor or hematopoietic malignancy.

In certain embodiments, the disease or condition being treated is hyperplasia.

In certain embodiments, the disease or condition being treated is a solid tumor.

In certain embodiments, the disease or condition being treated is a hematopoietic malignancy.

In certain embodiments, the subject being treated is further administered one or more of chemotherapy, radiotherapy, targeted therapy, immunotherapy or hormonal therapy.

In certain embodiments, the method comprises administering one or more of chemotherapy to the subject.

In certain embodiments, the method comprises administering a radiotherapy to the subject.

In certain embodiments, the method comprises administering a targeted therapy to the subject.

In certain embodiments, the method comprises administering an immunotherapy to the subject.

In certain embodiments, the method comprises administering hormonal therapy to the subject.

As used herein, the term “chemotherapeutic agent” refers to a chemical compound useful in the treatment of cancer. Examples of chemotherapeutic agents include Erlotinib (TARCEVA®, Genentech/OSI Pharm.), Bortezomib (VELCADE®, Millennium Pharm.), Fulvestrant (FASLODEX®, AstraZeneca), Sutent (SU11248, Pfizer), Letrozole (FEMARA®, Novartis), Imatinib mesylate (GLEEVEC®, Novartis), PTK787/ZK 222584 (Novartis), Oxaliplatin (Eloxatin®, Sanofi), 5-FU (5-fluorouracil), Leucovorin, Rapamycin (Sirolimus, RAPAMUNE®, Wyeth), Lapatinib (TYKERB®, GSK572016, Glaxo Smith Kline), Lonafarnib (SCH 66336), Sorafenib (BAY43-9006, Bayer Labs), and Gefitinib (IRESSA®, AstraZeneca), AG1478, AG1571 (SU 5271; Sugen), alkylating agents such as thiotepa and CYTOXAN® cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphoramide and trimethylomelamine; acetogenins (especially bullatacin and bullatacinone); a camptothecin (including the synthetic analog topotecan); bryostatin; callystatin; CC-1065 (including its adozelesin, carzelesin and bizelesin synthetic analogs); cryptophycins (particularly cryptophycin 1 and cryptophycin 8); dolastatin; duocarmycin (including the synthetic analogs, KW-2189 and CB1-TM1); eleutherobin; pancratistatin; a sarcodictyin; spongistatin; nitrogen mustards such as chlorambucil, chlornaphazine, chlorophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gammall and calicheamicin omegall (Angew Chem. Intl. Ed. Engl. (1994) 33: 183-186); dynemicin, including dynemicin A; bisphosphonates, such as clodronate; an esperamicin; as well as neocarzinostatin chromophore and related chromoprotein enediyne antibiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, ADRIAMYCIN® (doxorubicin), morpholino-doxorubicin, cyanomorpholino-doxorubicin, 2-pyrrolino-doxorubicin and deoxydoxorubicin), epirubicin, esonibicin, idarubicin, marcellomycin, mitomycins such as mitomycin C, mycophenolic acid, nogalamycin, olivomycins, peplomycin, porfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogs such as denopterin, methotrexate, pteropterin, trimetrexate; purine analogs such as fludarabine, 6-mercaptopurine, thiamniprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK® polysaccharide complex (JHS Natural Products, Eugene, Oreg.); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., TAXOL® (paclitaxel; Bristol-Myers Squibb Oncology, Princeton, N.J.), ABRAXANE® (Cremophor-free), albumin-engineered nanoparticle formulations of paclitaxel (American Pharmaceutical Partners, Schaumberg, 111.), and TAXOTERE® (doxetaxel; Rhone-Poulenc Rorer, Antony, France); chloranmbucil; GEMZAR® (gemcitabine); 6-thioguanine; mercaptopurine; methotrexate; platinum analogs such as cisplatin and carboplatin; vinblastine; etoposide (VP-16); ifosfamide; mitoxantrone; vincristine; NAVELBINE® (vinorelbine); novantrone; teniposide; edatrexate; daunomycin; aminopterin; capecitabine)(XELODA®; ibandronate; CPT-11; topoisomerase inhibitor RFS 2000; difluoromethylomithine (DMFO); retinoids such as retinoic acid; and pharmaceutically acceptable salts, acids and derivatives of any of the above.

Examples of the second (or further) agent or therapy may include, but are not limited to, immunotherapies (e.g. PD-1 inhibitors (pembrolizumab, nivolumab, cemiplimab), PD-L1 inhibitors (atezolizumab, avelumab, durvalumab), CTLA4 antagonist, cell signal transduction inhibitors (e.g., imatinib, gefitinib, bortezomib, erlotinib, sorafenib, sunitinib, dasatinib, vorinostat, lapatinib, temsirolimus, nilotinib, everolimus, pazopanib, trastuzumab, bevacizumab, cetuximab, ranibizumab, pegaptanib, panitumumab and the like), mitosis inhibitors (e.g., paclitaxel, vincristine, vinblastine and the like), alkylating agents (e.g., cisplatin, cyclophosphamide, chromabucil, carmustine and the like), anti-metabolites (e.g., methotrexate, 5-FU and the like), intercalating anticancer agents, (e.g., actinomycin, anthracycline, bleomycin, mitomycin-C and the like), topoisomerase inhibitors (e.g., irinotecan, topotecan, teniposide and the like), immunotherapic agents (e.g., interleukin, interferon and the like) and antihormonal agents (e.g., tamoxifen, raloxifene and the like).

In yet another aspect, the invention generally relates to use of a fusion protein or fusion protein complex, or prodrug thereof, disclosed herein for treating or reducing a disease or disorder (e.g., hyperplasia, solid tumor or hematopoietic malignancy).

In yet another aspect, the invention generally relates to use of a polynucleotide encoding a protein, such as a fusion protein or a fragment thereof, disclosed herein for treating or reducing a disease or disorder (e.g., hyperplasia, solid tumor or hematopoietic malignancy).

In yet another aspect, the invention generally relates to use of a fusion protein or fusion protein complex, or prodrug thereof, disclosed herein and a pharmaceutically acceptable excipient, carrier, or diluent, in preparation of a medicament for treating or reducing a disease or disorder (e.g., hyperplasia, solid tumor or hematopoietic malignancy).

In certain embodiments, the disease or disorder that may be treated with the fusion protein or fusion protein complex, or prodrug thereof, include one or more selected from head and neck cancer, endometrial cancer, colorectal cancer, ovarian cancer, breast cancer, melanoma, lung cancer, renal cancer, liver cancer, anal cancer, sarcoma, lymphoma, leukemia, brain tumors, gastric cancer, testicular cancer, pancreatic cancer, and thyroid cancer. Exemplary disease or disorder include acute myeloid leukemia, adrenocortical carcinoma. B-cell lymphoma, bladder urothelial carcinoma, breast ductal carcinoma, breast lobular carcinoma, carcinomas of the esophagus, castration-resistant prostate cancer (CRPC), cervical carcinoma, cholangiocarcinoma, chronic myelogenous leukemia, colorectal adenocarcinoma, colorectal cancer (CRC), esophageal carcinoma, gastric adenocarcinoma, glioblastoma multiforme, head and neck squamous cell carcinoma, Hodgkin's lymphoma/primary mediastinal B-cell lymphoma, hepatocellular carcinoma (HCC), kidney chromophobe carcinoma, kidney clear cell carcinoma, kidney papillary cell carcinoma, lower grade glioma, lung adenocarcinoma, lung squamous cell carcinoma, melanoma (MEL), mesothelioma, non-squamous NSCLC, ovarian serous adenocarcinoma, pancreatic ductal adenocarcinoma, paraganglioma & pheochromocytoma, prostate adenocarcinoma, renal cell carcinoma (RCC), sarcoma, skin cutaneous melanoma, squamous cell carcinoma of the head and neck, T-cell lymphoma, thymoma, thyroid papillary carcinoma, uterine carcinosarcoma, uterine corpus endometrioid carcinoma and uveal melanoma.

In certain embodiments, the disease or disorder that may be treated with the fusion protein or fusion protein complex, or prodrug thereof, is B-cell lymphoma.

In certain embodiments, the disease or disorder that may be treated with the fusion protein or fusion protein complex, or prodrug thereof, is B colorectal cancer.

Methods and use of the invention may be a combination therapy with the herein disclosed agents being used in combination with one or more of immune check point blockade, co-signaling of T cells, and tumor targeting antibody therapies.

In yet another aspect, the invention generally relates to a cell line comprising a polynucleotide encoding a fusion protein or fusion protein complex, or prodrug thereof, disclosed herein.

In yet another aspect, the invention generally relates to a method for making a protein, comprising culturing the cell line. In certain embodiments, the method further comprises purifying or isolating a produced protein, such as a fusion protein or fusion protein complex, or prodrug thereof disclosed herein.

In yet another aspect, the invention generally relates to a method for making a protein. The method comprises: providing an expression vector encoding a fusion protein or fusion protein complex, or prodrug thereof disclosed herein; introducing into a host cell the expression vector; culturing the host cell in media under conditions sufficient to express the protein; and purifying the protein from the host cell or media.

In certain embodiments, the method comprises constructing an expression vector comprising the coding gene encoding a fusion protein or prodrug; constructing the host cell comprising the expression vector by transiently transfection; culturing the host cell; collecting the cell supernatant; and purifying the fusion protein or prodrug by affinity chromatography of Protein A/G.

Any suitable expression vectors may be employed.

Any suitable host cell may be employed, for examples, 293F and CHO cells.

Introduction of the expression vector can be accomplished by any suitable transfection method and can be via a transient transfection or a stable cell line.

Any suitable purification method may be employed. An exemplary purification method is by affinity chromatography of ProteinA/G or size exclusion methods.

In yet another aspect, the invention generally relates to an isolated protein produced by a method disclosed herein.

In certain embodiments, the isolated protein is substantially pure.

As disclosed herein, linker sequences can be used to link two or more polypeptides of the biologically active polypeptide to generate a single-chain molecule with a desired functional activity.

Any suitable linkers may be adopted. Exemplary peptide linker sequences include those having range from about 7 to 28 amino acids, e.g., from about 8 to 16 amino acids. The linker sequence is preferably flexible so as not hold the biologically active polypeptide or effector molecule in a single undesired conformation. The linker sequence can be used, e.g., to space the recognition site from the fused molecule. Specifically, the peptide linker sequence can be positioned so as to provide molecular flexibility. The linker preferably predominantly comprises amino acids with small side chains, such as glycine, alanine and serine, to provide for flexibility.

In general, preparation of the fusion protein complexes of the invention can be accomplished by procedures disclosed herein and by recognized recombinant DNA techniques involving, e.g., polymerase chain amplification reactions (PCR), preparation of plasmid DNA, cleavage of DNA with restriction enzymes, preparation of oligonucleotides, ligation of DNA, isolation of mRNA, introduction of the DNA into a suitable cell, transformation or transfection of a host, culturing of the host. Additionally, the fusion molecules can be isolated and purified using chaotropic agents and well known electrophoretic, centrifugation and chromatographic methods. (Sambrook, et al., Molecular Cloning: A Laboratory Manual (2nd ed. (1989); and Ausubel, et al., Current Protocols in Molecular Biology, John Wiley & Sons, New York (1989) for disclosure relating to these methods.)

The invention further provides nucleic acid sequences and DNA sequences that encode the present fusion proteins. The DNA sequence may be carried by a vector suited for extrachromosomal replication such as a phage, virus, plasmid, phagemid, cosmid, YAC, or episome. For example, a DNA vector that encodes a desired fusion protein can be used to facilitate preparative methods described herein and to obtain significant quantities of the fusion protein or components thereof. The DNA sequence can be inserted into an appropriate expression vector, i.e., a vector that contains the necessary elements for the transcription and translation of the inserted protein-coding sequence. A variety of host-vector systems may be utilized to express the protein-coding sequence. These may include mammalian cell systems infected with virus (e.g., vaccinia virus, adenovirus, etc.); insect cell systems infected with virus (e.g., baculovirus); microorganisms such as yeast containing yeast vectors, or bacteria transformed with bacteriophage DNA, plasmid DNA or cosmid DNA. Depending on the host-vector system utilized, any one of a number of suitable transcription and translation elements may be used. (Sambrook, et al., Molecular Cloning: A Laboratory Manual (2nd ed. (1989); and Ausubel, et al., Current Protocols in Molecular Biology, John Wiley & Sons, New York (1989) for disclosure relating to these methods.)

Fusion protein components encoded by the DNA vector can be provided in a cassette format. By the term “cassette” is meant that each component can be readily substituted for another component by standard recombinant methods. In particular, a DNA vector configured in a cassette format is particularly desirable when the encoded fusion complex is to be used against pathogens that may have or have capacity to develop serotypes.

To make the vector coding for a fusion protein complex, the sequence coding for the biologically active polypeptide is linked to a sequence coding for the effector peptide by use of suitable ligases. DNA coding for the presenting peptide can be obtained by isolating DNA from natural sources such as from a suitable cell line or by known synthetic methods, e.g. the phosphate triester method. (Oligonucleotide Synthesis, IRL Press, M. J. Gait, ed., 1984). Synthetic oligonucleotides also may be prepared using commercially available automated oligonucleotide synthesizers. Once isolated, the gene coding for the biologically active polypeptide can be amplified by PCR or other means known in the art. Suitable PCR primers to amplify the biologically active polypeptide gene may add restriction sites to the PCR product. The PCR product preferably includes splice sites for the effector peptide and leader sequences necessary for proper expression and secretion of the biologically active polypeptide-effector fusion complex. The PCR product also preferably includes a sequence coding for the linker sequence, or a restriction enzyme site for ligation of such a sequence.

The fusion proteins described herein may be produced by standard recombinant DNA techniques. For example, once a DNA molecule encoding the biologically active polypeptide is isolated, sequence can be ligated to another DNA molecule encoding the effector polypeptide. The nucleotide sequence coding for a biologically active polypeptide may be directly joined to a DNA sequence coding for the effector peptide or, more typically, a DNA sequence coding for the linker sequence as discussed herein may be interposed between the sequence coding for the biologically active polypeptide and the sequence coding for the effector peptide and joined using suitable ligases. The resultant hybrid DNA molecule can be expressed in a suitable host cell to produce the fusion protein complex. The DNA molecules are ligated to each other in a 5′ to 3′ orientation such that, after ligation, the translational frame of the encoded polypeptides is not altered (i.e., the DNA molecules are ligated to each other in-frame). The resulting DNA molecules encode an in-frame fusion protein.

Other nucleotide sequences also can be included in the gene construct. For example, a promoter sequence, which controls expression of the sequence coding for the biologically active polypeptide fused to the effector peptide, or a leader sequence, which directs the fusion protein to the cell surface or the culture medium, can be included in the construct or present in the expression vector into which the construct is inserted.

In obtaining variant biologically active polypeptide, IL15, IL15R or Fc domain coding sequences, those of ordinary skill in the art will recognize that the polypeptides may be modified by certain amino acid substitutions, additions, deletions, and post-translational modifications, without loss or reduction of biological activity. In particular, it is well-known that conservative amino acid substitutions, that is, substitution of one amino acid for another amino acid of similar size, charge, polarity and conformation, are unlikely to significantly alter protein function. The 20 standard amino acids that are the constituents of proteins can be broadly categorized into four groups of conservative amino acids as follows: the nonpolar (hydrophobic) group includes alanine, isoleucine, leucine, methionine, phenylalanine, proline, tryptophan and valine; the polar (uncharged, neutral) group includes asparagine, cysteine, glutamine, glycine, serine, threonine and tyrosine; the positively charged (basic) group contains arginine, histidine and lysine; and the negatively charged (acidic) group contains aspartic acid and glutamic acid. Substitution in a protein of one amino acid for another within the same group is unlikely to have an adverse effect on the biological activity of the protein. In other instance, modifications to amino acid positions can be made to reduce or enhance the biological activity of the protein. Such changes can be introduced randomly or via site-specific mutations based on known or presumed structural or functional properties of targeted residue(s). Following expression of the variant protein, the changes in the biological activity due to the modification can be readily assessed using binding or functional assays.

Homology between nucleotide sequences can be determined by DNA hybridization analysis, wherein the stability of the double-stranded DNA hybrid is dependent on the extent of base pairing that occurs. Conditions of high temperature and/or low salt content reduce the stability of the hybrid, and can be varied to prevent annealing of sequences having less than a selected degree of homology. For instance, for sequences with about 55% G-C content, hybridization and wash conditions of 40-50 C, 6×SSC (sodium chloride/sodium citrate buffer) and 0.1% SDS (sodium dodecyl sulfate) indicate about 60-70% homology, hybridization and wash conditions of 50-65 C, 1×SSC and 0.1% SDS indicate about 82-97% homology, and hybridization and wash conditions of 52 C, 0.1×SSC and 0.1% SDS indicate about 99-100% homology. A wide range of computer programs for comparing nucleotide and amino acid sequences (and measuring the degree of homology) are also available. Readily available sequence comparison and multiple sequence alignment algorithms are, respectively, the Basic Local Alignment Search Tool (BLAST) and ClustalW programs.

A number of strategies can be employed to express protein fusion complexes of the invention. For example, the fusion protein construct described above can be incorporated into a suitable vector by known means such as by use of restriction enzymes to make cuts in the vector for insertion of the construct followed by ligation. The vector containing the gene construct is then introduced into a suitable host for expression of the fusion protein. (Sambrook et al., Molecular Cloning: A Laboratory Manual (2nd ed. (1989) for disclosure relating to these methods.)

Selection of suitable vectors can be made empirically based on factors relating to the cloning protocol. For example, the vector should be compatible with, and have the proper replicon for the host that is being employed. Further the vector must be able to accommodate the DNA sequence coding for the fusion protein complex that is to be expressed. Suitable host cells include eukaryotic and prokaryotic cells, preferably those cells that can be easily transformed and exhibit rapid growth in culture medium. Specifically, preferred hosts cells include prokaryotes such as E. coli, Bacillus subtillus, etc. and eukaryotes such as animal cells and yeast strains, e.g., S. cerevisiae. Mammalian cells are generally preferred, particularly J558, NSO, SP2-O or CHO. Other suitable hosts include, e.g., insect cells such as Sf9. Conventional culturing conditions are employed. See Sambrook, supra. Stable transformed or transfected cell lines can then be selected. Cells expressing a fusion protein complex of the invention can be determined by known procedures. For example, expression of a fusion protein complex linked to an immunoglobulin can be determined by an ELISA specific for the linked immunoglobulin and/or by immunoblotting. Other methods for detecting expression of fusion proteins comprising biologically active polypeptides linked to IL15 or IL15R domains are disclosed in the Examples.

A host cell can be used for preparative purposes to propagate nucleic acid encoding a desired fusion protein or a component thereof. A host cell can include a prokaryotic or eukaryotic cell in which production of the fusion protein is specifically intended. Thus, host cells specifically include yeast, fly, worm, plant, frog, mammalian cells and organs that are capable of propagating nucleic acid encoding the fusion. Non-limiting examples of mammalian cell lines which can be used include CHO dhfr-cells (Urlaub and Chasm, 1980 Proc. Natl. Acad. Sci. USA, 77:4216), 293 cells (Graham et al. 1977 J. Gen. Virol., 36:59 ( )) or myeloma cells like SP2 or NSO (Galfre and Milstein, 1981 Meth. Enzymol., 73(B):3).

Host cells capable of propagating nucleic acid encoding a desired fusion protein complex encompass non-mammalian eukaryotic cells as well, including insect (e.g., Sp. frugiperda), yeast (e.g., S. cerevisiae, S. pombe, P. pastoris, K lactis, H. polymorpha; as generally reviewed by Fleer, R., 1992 Current Opinion in Biotechnology, 3(5):486496), fungal and plant cells. Also contemplated are certain prokaryotes such as E. coli and Bacillus.

Nucleic acid encoding a desired fusion protein can be introduced into a host cell by standard techniques for transfecting cells. The term “transfecting” or “transfection” is intended to encompass all conventional techniques for introducing nucleic acid into host cells, including calcium phosphate co-precipitation, DEAE-dextran-mediated transfection, lipofection, electroporation, microinjection, viral transduction and/or integration.

Various promoters (transcriptional initiation regulatory region) may be used according to the invention. The selection of the appropriate promoter is dependent upon the proposed expression host. Promoters from heterologous sources may be used as long as they are functional in the chosen host.

Promoter selection is also dependent upon the desired efficiency and level of peptide or protein production. Inducible promoters such as tac are often employed in order to dramatically increase the level of protein expression in E. coli. Overexpression of proteins may be harmful to the host cells. Consequently, host cell growth may be limited. The use of inducible promoter systems allows the host cells to be cultivated to acceptable densities prior to induction of gene expression, thereby facilitating higher product yields.

Various signal sequences may be used according to the invention. A signal sequence which is homologous to the biologically active polypeptide coding sequence may be used. Alternatively, a signal sequence which has been selected or designed for efficient secretion and processing in the expression host may also be used. A signal sequence may be joined directly through the sequence encoding the signal peptidase cleavage site to the protein coding sequence, or through a short nucleotide bridge.

The expression construct can be assembled by employing known recombinant DNA techniques. Restriction enzyme digestion and ligation are the basic steps employed to join two fragments of DNA. Polylinkers and adaptors may be employed to facilitate joining of selected fragments. The expression construct can typically be assembled in stages employing rounds of restriction, ligation, and transformation of E. coli. Numerous cloning vectors suitable for construction of the expression construct are known in the art (λZAP and pBLUESCRIPT SK-1, Stratagene, La Jolla, Calif., pET, Novagen Inc., Madison, Wis., pEE12.4, Lonza Biologics, Basel, Switzerland).

The expression construct may be transformed into the host as the cloning vector construct, either linear or circular, or may be removed from the cloning vector and used as is or introduced onto a delivery vector. The delivery vector facilitates the introduction and maintenance of the expression construct in the selected host cell type. The expression construct is introduced into the host cells by any of a number of known gene transfer systems (e.g., natural competence, chemically mediated transformation, protoplast transformation, electroporation, biolistic transformation, transfection, or conjugation). The gene transfer system selected depends upon the host cells and vector systems used.

The present invention further provides a production process for isolating a fusion protein of interest. In the process, a host cell (e.g., a yeast, fungus, insect, bacterial or animal cell), into which has been introduced a nucleic acid encoding the protein of the interest operatively linked to a regulatory sequence, is grown at production scale in a culture medium to stimulate transcription of the nucleotides sequence encoding the fusion protein of interest. Subsequently, the fusion protein of interest is isolated from harvested host cells or from the culture medium. Standard protein purification techniques can be used to isolate the protein of interest from the medium or from the harvested cells. In particular, the purification techniques can be used to express and purify a desired fusion protein on a large-scale (i.e. in at least milligram quantities) from a variety of implementations including roller bottles, spinner flasks, tissue culture plates, bioreactor, or a fermentor.

An expressed protein fusion complex can be isolated and purified by known methods. Typically, the culture medium is centrifuged or filtered and then the supernatant is purified by affinity or immunoaffinity chromatography, e.g. Protein-A or Protein-G affinity chromatography or an immunoaffinity protocol comprising use of monoclonal antibodies that bind the expressed fusion complex such as a linked TCR or immunoglobulin region thereof. The fusion proteins of the present invention can be separated and purified by appropriate combination of known techniques. These methods include, for example, methods utilizing solubility such as salt precipitation and solvent precipitation, methods utilizing the difference in molecular weight such as dialysis, ultra-filtration, gel-filtration, and SDS-polyacrylamide gel electrophoresis, methods utilizing a difference in electrical charge such as ion-exchange column chromatography, methods utilizing specific affinity such as affinity chromatography, methods utilizing a difference in hydrophobicity such as reverse-phase high performance liquid chromatography and methods utilizing a difference in isoelectric point, such as isoelectric focusing electrophoresis, metal affinity columns such as Ni-NTA. (Sambrook et al., Molecular Cloning: A Laboratory Manual (2nd ed. (1989); and Ausubel et al., Current Protocols in Molecular Biology, John Wiley & Sons, New York (1989) for disclosure relating to these methods.)

It is preferred that the fusion proteins of the present invention be substantially pure. That is, the fusion proteins have been isolated from cell substituents that naturally accompany it so that the fusion proteins are present preferably in at least 80% or 90% to 95% homogeneity (w/w). Fusion proteins having at least 98 to 99% homogeneity (w/w) are most preferred for many pharmaceutical, clinical and research applications. Once substantially purified the fusion protein should be substantially free of contaminants for therapeutic applications. Once purified partially or to substantial purity, the soluble fusion proteins can be used therapeutically, or in performing in vitro or in vivo assays as disclosed herein. Substantial purity can be determined by a variety of standard techniques such as chromatography and gel electrophoresis.

The invention also provides a pharmaceutical preparation comprising a therapeutically effective amount of a composition, a fusion protein, a polynucleotide, a gene construct, a vector or a host cell according to the invention and a pharmaceutically acceptable excipient or vehicle.

Preferred excipients for use in the present invention include sugars, starches, celluloses, gums and proteins. In a preferred embodiment, the pharmaceutical composition of the invention is formulated in a pharmaceutical form for administration as a solid (for example tablets, capsules, lozenges, granules, suppositories, crystalline or amorphous sterile solids that can be reconstituted to provide liquid forms, etc.), liquid (for example solutions, suspensions, emulsions, elixirs, lotions, unguents, etc.) or semi-solid (gels, ointments, creams and similar). The pharmaceutical compositions of the invention can be administered by any route, including, without limitation, oral, intravenous, intramuscular, intraarterial, intramedullary, intratecal, intraventricular, transdermic, subcutaneous, intraperitoneal, intranasal, enteric, topical, sublingual or rectal route. A revision of the different forms of administration of active principles, the excipients to be used and their manufacturing procedures can be found in Remington's Pharmaceutical Sciences (A. R. Gennaro, Ed.), 20^{th} edition, Williams & Wilkins PA, USA (2000) Examples of pharmaceutically acceptable vehicles are known in the state of the technique and include saline solutions buffered with phosphate, water, emulsions, such as oil/water emulsions, different types of humidifying agents, sterile solutions, etc. The compositions comprising said vehicles can be formulated by conventional procedures known in the state of the technique.

In the case of the pharmaceutical composition of the invention comprising nucleic acids (the polynucleotides of the invention, vectors or gene constructs), the invention contemplates specially prepared pharmaceutical compositions for administering said nucleic acids. The pharmaceutical compositions can comprise said nucleic acids in naked form, in other words, in the absence of compounds protecting the nucleic acids from degradation by the organism's nucleases, which entails the advantage of eliminating the toxicity associated to the reagents used for transfection. Suitable routes of administration for the naked compounds include intravascular, intratumoral, intracraneal, intraperitoneal, intrasplenic, intramuscular, subretinal, subcutaneous, mucous, topical and oral route (Templeton, 2002 DNA Cell Biol., 21:857-867). Alternatively, the nucleic acids can be administered forming part of liposomes, conjugated to cholesterol or conjugated to compounds capable of promoting translocation through cell membranes such as the Tat peptide derived from the TAT protein of HIV-1, the third helix of the homeodomain of the Antennapedia protein of D. melanogaster, the VP22 protein of the herpes simplex virus, oligomers of arginine and peptides such as those described in WO07069090 (Lindgren, et al. 2000 Trends Pharmacol. Sci 21:99-103; Schwarze, et al. 2000 Trends Pharmacol. Sci. 21:45-48; Lundberg, et al. 2003 Mol. Therapy 8:143-150; and Snyder, et al. 2004 Pharm. Res. 21:389-393). Alternatively, the polynucleotide can be administered forming part of a plasmidic vector or of a viral vector, preferably vectors based on an adenovirus, in adeno-associated viruses or in retroviruses, such as viruses based on the virus of murine leukaemia (MLN) or on lentiviruses (HIV, FIV, EIAV).

The compositions of the invention can be administered at doses of less than 10 mg per kilogram of body weight, preferably less than 5, 2, 1, 0.5, 0.1, 0.05, 0.01, 0.005, 0.001, 0.0005, 0.0001, 0.00005 or 0.00001 mg per each kg of body weight and less than 200 nmol of agent, in other words, approximately 4.4×10¹⁶ copies per kg of body weight or less than 1500, 750, 300, 150, 75, 15, 7.5, 1.5, 0.75, 0.15 or 0.075 nmol per Kg of body weight. The unitary dose can be administered by injection, by inhalation or by topical administration. The bifunctional polynucleotides and compositions of the invention can be administered directly into the organ in which the target mRNA is expressed in which case doses will be administered of between 0.00001 mg and 3 mg per organ, or preferably between 0.0001 and 0.001 mg per organ, about 0.03 and 3.0 mg per organ, about 0.1 and 3.0 mg per organ or between 0.3 and 3.0 mg per organ.

The dose will depend on the severity and response to the condition to be treated and may vary between several days and several months or until the condition is seen to remit. The optimum dose can be determined by periodically measuring the agent's concentrations in the patient's organism. The optimum dose can be determined from the EC50 values obtained through previous in vitro or in vivo tests in animal models. The unitary dose can be administered once a day or less than once a day, preferably, less than once every 2, 4, 8 or 30 days. Alternatively, it is possible to administer an initial dose followed by one or several maintenance doses, generally in a lesser amount that the initial dose. The maintenance regime may involve treating the patient with doses ranging between 0.01 μg and 1.4 mg/kg of body weight per day, for example, 1, 0.1, 0.01, 0.001, or 0.00001 mg per kg of body weight per day. Maintenance doses are administered, preferably, at most once every 5, 10 or 30 days. The treatment must continue for a time that will vary according to the type of alteration suffered by the patient, its severity and the patient's condition. Following treatment, the patient's evolution must be monitored in order to determine whether the dose ought to be increased in the case of the disease not responding to the treatment or whether the dose ought to be decreased in the case of observing an improvement in the disease or unwanted secondary effects.

The daily dose can be administered in a single dose or in two or more doses according to the particular circumstances. If a repeated administration or frequent administrations are required, it is advisable to implant an administration device, such as a pump, a semi-permanent catheter (intravenous, intraperitoneal, intracisternal or intracapsular) or a reservoir.

The compositions of the invention are administered according to methods known to an expert in the art, including, without limitation, intravenous, oral, nasal, parenteral, topical, transdermic, rectal and similar.

The following examples are meant to be illustrative of the practice of the invention and not limiting in any way.

### Examples

The below Examples describe certain exemplary embodiments of compounds prepared according to the disclosed invention. It will be appreciated that the following general methods, and other methods known to one of ordinary skill in the art, can be applied to compounds and subclasses and species thereof, as disclosed herein.

Poor Tumor Control and Strong Toxicity after Systemic Delivery of IL15-Rα-Fc

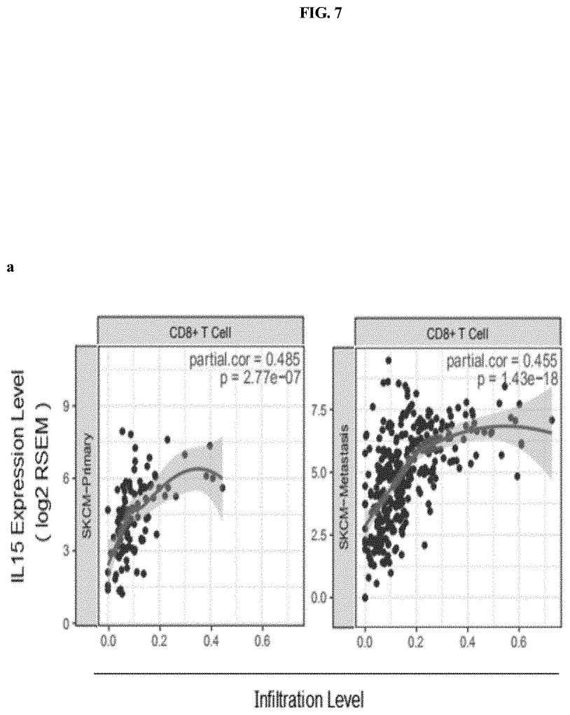

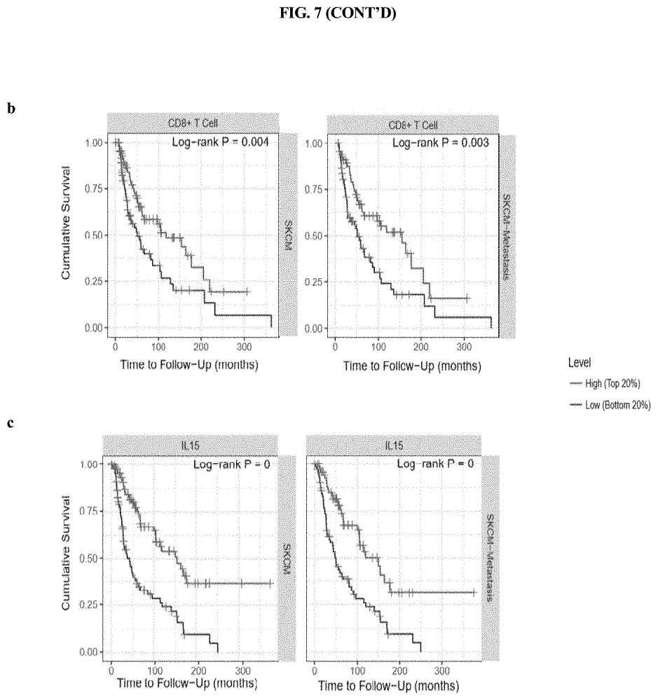

We proposed that insufficient IL-15 secretion within tumor may be one cause of failure T cell mediated tumor growth. In human skin melanoma cancer samples, high CD8⁺ T cells infiltration was always correlated with high IL-15 level (FIG. 7A). Moreover, both CD8⁺ T cell quantity and IL-15 expression level within tumor positively correlate with better survival in human cancer patients (FIGS. 7B and 7C). These data suggested that IL-15 might contribute to CD8⁺ T cells infiltration and tumor inhibition.

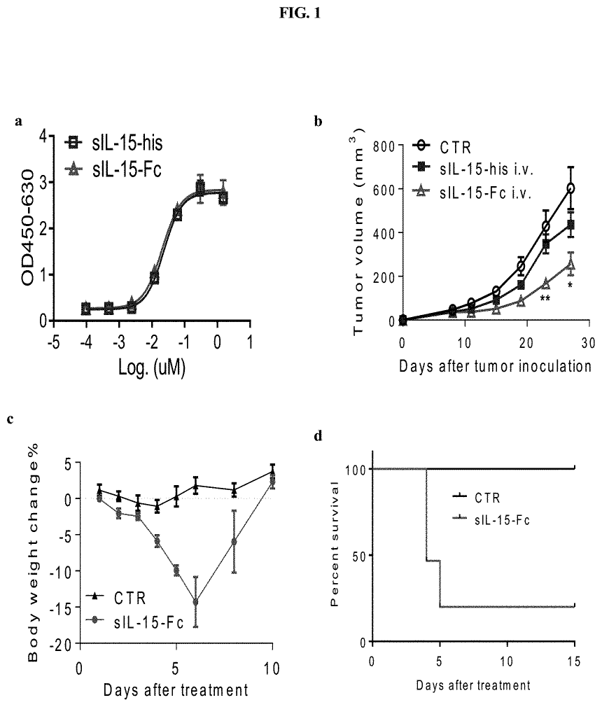

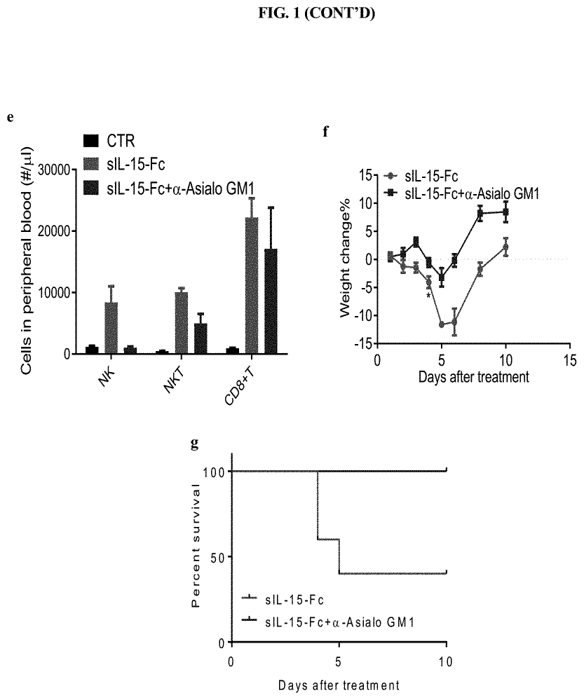

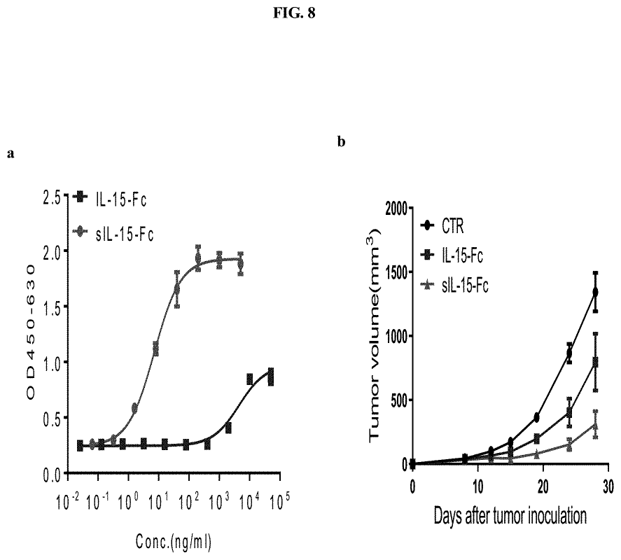

Consequently, we explored whether providing additional IL-15 to expand T cells could improve tumor control. In the light of the action mechanism of trans-presentation for IL-15, it is reported that IL-15 fused with IL-15Rαsushi domain (super IL-15) have much increased activity and stability than IL-15 alone. Fc fusion is reported to increase half-life in vivo and binding affinity through dimerization. Together, we designed and generated IL-15-Fc and IL-15-IL15Rα-Fc (Super IL-15-Fc or sIL-15-Fc) protein. sIL-15-Fc have 100-fold increased activity than IL-15-Fc protein, as in vitro CTLL2 expansion assay showed (FIG. 8A). Consistently, sIL-15-Fc have much better tumor control effect than IL-15-Fc in A20 model (FIG. 8B). Though the in vitro activity is similar, sIL-15-Fc had much better tumor control than sIL-15-Fc-his, suggesting the Fc fusion indeed increase its in vivo stability (FIGS. 1A and 1B). In both clinical and experimental animal tumor models, IL-15 treatment alone showed rather limited efficacy but significant toxicity in therapeutic doses. Consistently, we observed super IL-15-Fc could only partially control tumor growth in the A20 tumor model (FIG. 1B). When the dose increased, severe systemic toxicity become obvious, including body-weight lost and decreased survival (FIGS. 1C and 1D).

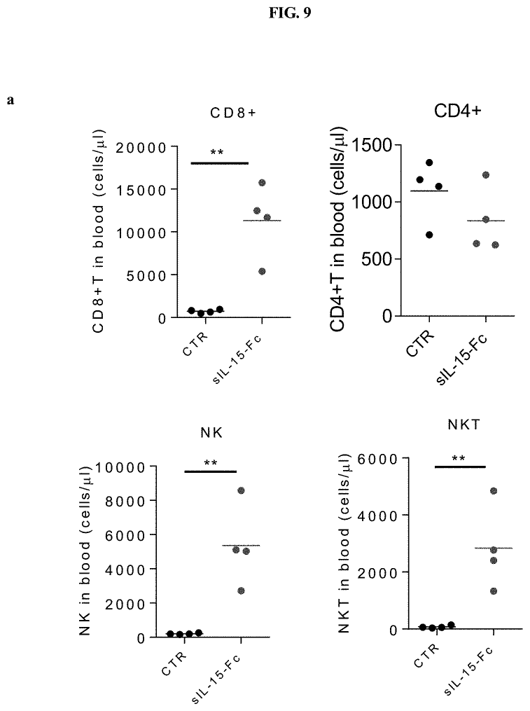

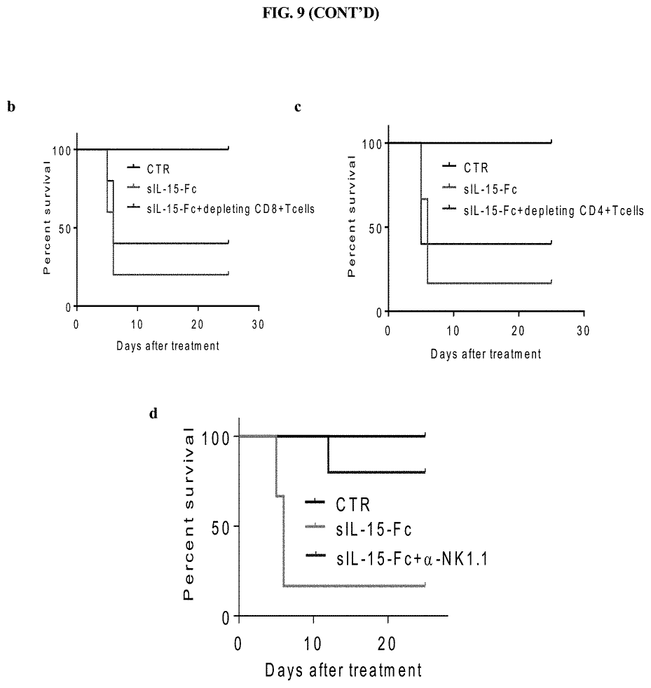

After super IL-15-Fc treatment, CD8⁺ T cells, NK and NKT cells were found significantly increased in the peripheral blood (FIG. 9A). The quantity of CD4⁺ T cells was not affected after super IL-15-Fc treatment (FIG. 9A). Though CD8⁺ T cells and NK cells were reported to contribute to the tumor inhibition, the systemic activation of various T cells and NK cells can result in off-tumor toxicities. To explore which immune cells mediated toxicity, we used antibodies to deplete CD8⁺ T, NK, NKT or CD4⁺ T separately. The depletion of CD8⁺ T or CD4⁺ T cells did not affect the survival of sIL-15-Fc treated mice, suggesting CD8⁺ T or CD4⁺ T cells are not essential (FIGS. 9B and 9C). However, administration of anti-NK1.1 antibody prevented the mice dying from the sIL-15-Fc treatment (FIG. 9D). Anti-NK1.1 (clone PK136) antibody could deplete both NK and NKT. To further disseminate whether NK or NKT is detrimental, we used anti-asialo GM1 antibody to deplete NK only but not NKT (FIG. 1C). The depletion of only NK completely protected mice, suggesting that NK cells are main cellular components for the sIL-15-Fc induced toxicity (FIGS. 1F and 1G). These data suggested expansion of peripheral NK mediated by systemic administrated sIL-15-Fc induced off-tumor toxicity, which should be carefully considered in the clinic.

### Engineering a Tumor-Conditional Pro-IL15

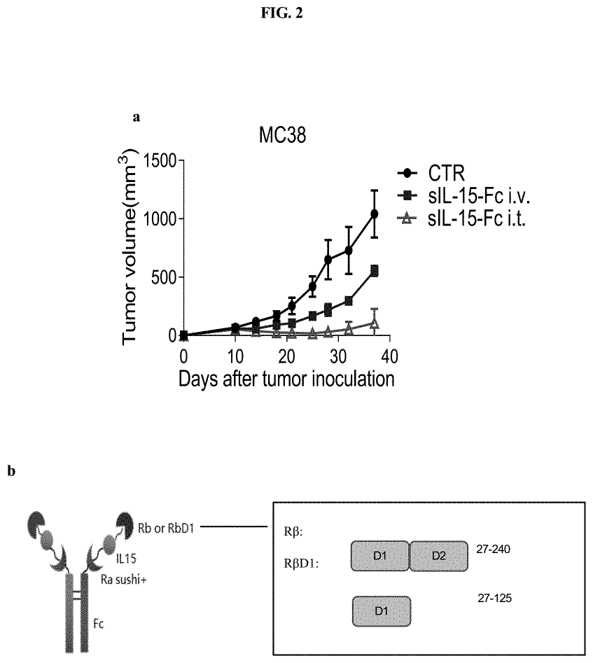

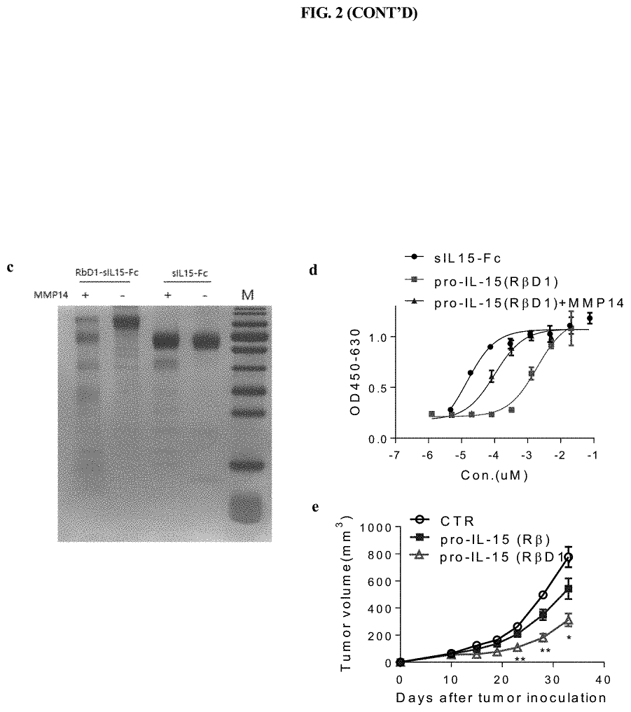

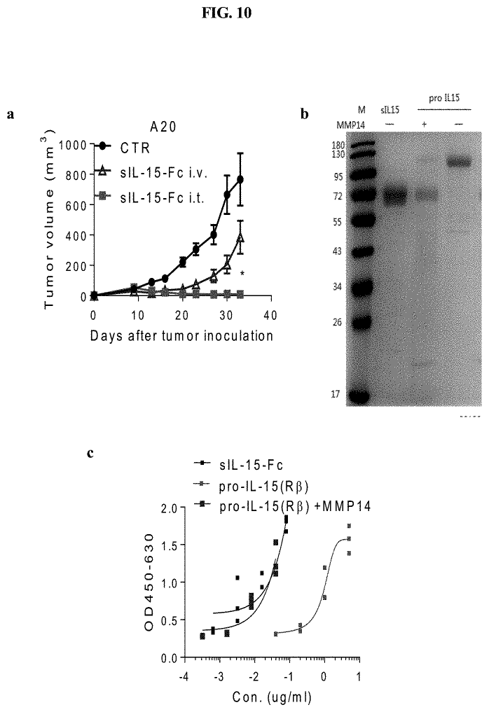

Locally delivered super-IL-15-Fc by intratumoral injection harvested better tumor control effect than systemic treatment in both MC38 and A20 tumor model (FIG. 2A and FIG. 10A). These data suggest that sIL-15-Fc might be more potent against tumors with limited toxicity if targeting delivery into the TME. To design IL-15 that can be preferentially activated inside TME, we created pro-IL-15, during which the extracellular domain of IL15Rβ is fused into the N-terminal of sIL-15-Fc by an MMP-14 cleavable peptide linker. In contrast to many MMPs that are soluble and easily leak out systemically, MMP-14 is a membrane protein and is highly expressed on tumor cells and tumor-associated macrophages and stayed within tumor tissues. We choose a very sensitive linker that could be cleaved by MMP-14 protease, acting as switch to expose IL-15 in MMP enriched tumor microenvironment (FIG. 2B). To optimize the release of active IL-15 after the linker cleavage, we also designed binding decreased extracellular domain of IL15Rβ (RβD1) for pro-IL15 (FIG. 2B).

Reducing SDS-PAGE results showed that, the MW of monomeric sIL15-Fc is about 65 Kd, and pro-IL-15 is about 85 Kd, which are larger than expected and possibly caused by glycosylation (FIG. 2C and FIG. 10B). After incubation with MMP-14, pro-IL-15 was cut into sIL-15-Fc (FIG. 2C and FIG. 10B). Then we compared the bioactivity of pro-IL-15 before and after cutting through CTLL-2 proliferation assay. sIL-15-Fc could proliferate CTLL-2 cells in a dose dependent manner (FIG. 2D). Pro-IL-15 with Rβ have 100-fold decreased activity than super-IL-15, while pro-IL-15 with ROM have about 50-fold decreased activity (FIG. 2D for ROM and FIG. 10C for Rβ). After incubation with MMP-14, the activity of both two forms of pro-IL-15 was recovered significantly comparable to sIL-15-Fc. In syngeneic MC38 tumor model, pro-IL-15 with ROD′ inhibit tumor growth much better than which with Rβ(D1+D2), suggesting the former have optimized activity in vivo (FIG. 2E).

### Pro-IL15 Avoids Peripheral NK Expansion Mediated Toxicity

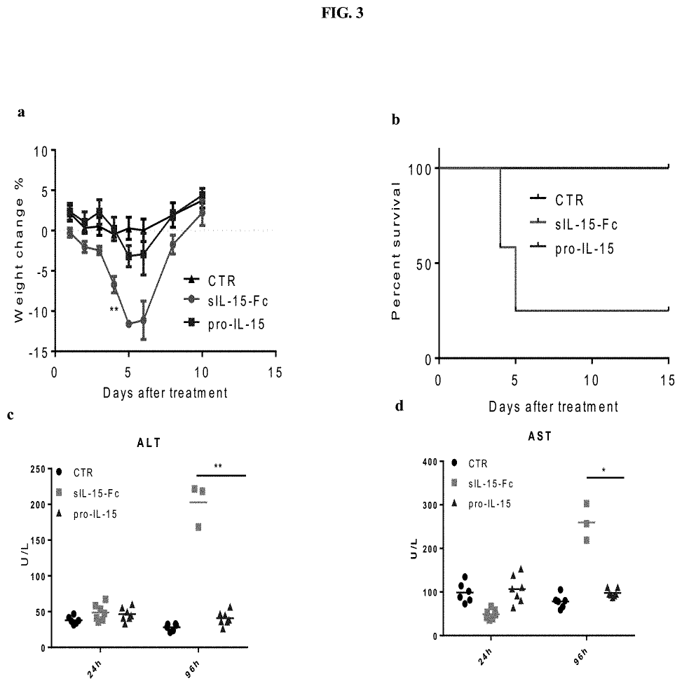

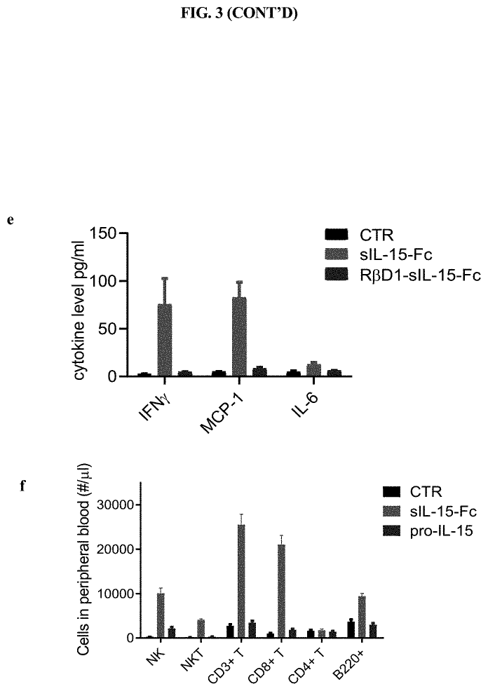

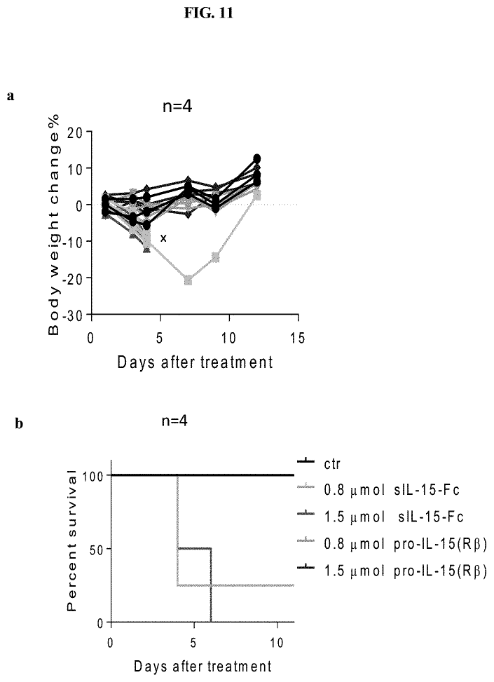

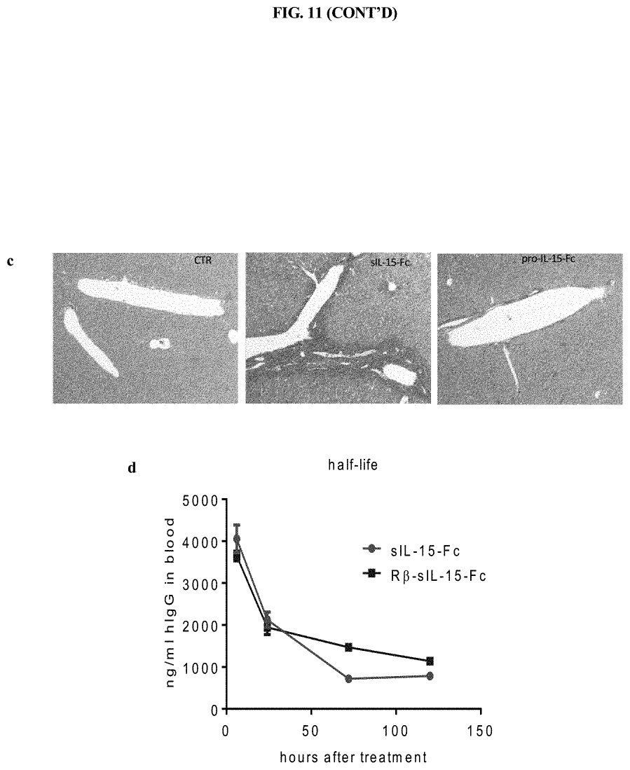

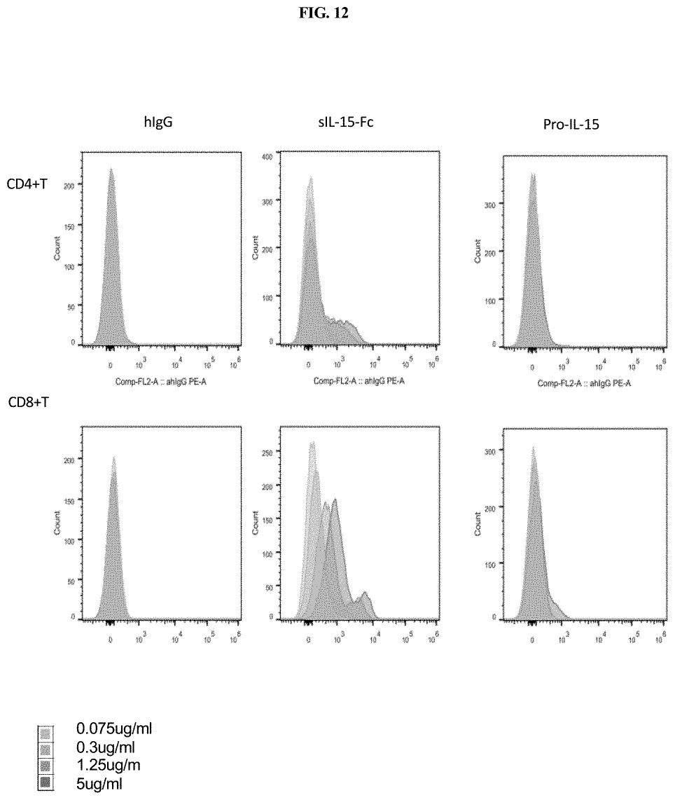

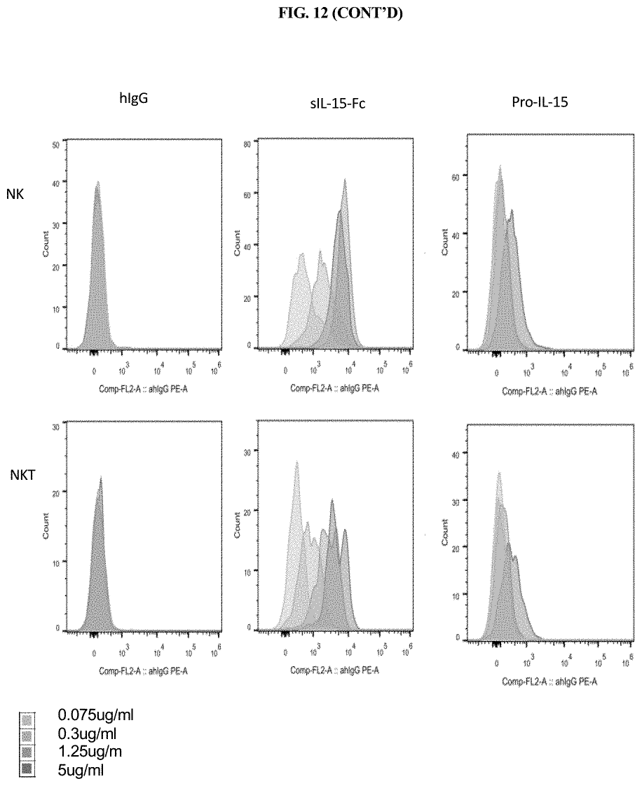

We proposed that the design of pro-IL-15 will avoid the expansion of peripheral lymphocytes and induce less toxicity. To confirm this, we administrated lethal dose of sIL-15-Fc or comparable molecular of pro-IL-15 into mice. 80 μg of sIL-15-Fc induced significant body-weight lost, and eventually 70% of mice die at about 5 days after treatment (FIG. 3A). However, pro-IL-15 treated mice recovered to normal after a transient slightly body-weight lost, and no mice died (FIGS. 3A and 3B). To further characterize the toxicity that is induced by cytokine treatment, we performed blood chemistry analysis about alanine aminotransferase (ALT, a liver damage marker) and aspartate aminotransferase (AST, a tissue damage marker) 24 h and 96 h after treatment in tumor-bearing mice. sIL-15-Fc increased both serum ALT and AST activity, but pro-IL-15 did not (FIGS. 3C and 3D). sIL-15-Fc increased serum inflammatory cytokines such as IFN-γ, MCP-1 and IL-6, but pro-IL-15 did not induce inflammatory cytokines (FIG. 3E). Consistently, in contrast to sIL-15-Fc induced sharp increase of NK cells in peripheral blood, pro-IL-15 induced much less NK expansion (FIG. 3F). The expansion of peripheral lymphocytes may impair peripheral solid organ such as liver tissue. Indeed, sIL-15-Fc induced significant immune cells infiltration into liver tissue (FIG. 11C). However, pro-IL-15 treatment induced no liver inflammation, showed normally as control mice (FIG. 11C). The serum half-life of pro-IL-15 and sIL-15-Fc is similar, suggesting the decreased toxicity of peripheral pro-IL-15 is not caused by protein instability (FIG. 11D). To confirm the decreased bioactivity of pro-IL-15 in peripheral tissues, we compared their binding ability with lymphocytes. sIL-15-Fc showed strong binding with splenic NK, NKT and CD8⁺ T cells, while little binding with CD4⁺ T cells (FIG. 12). However, pro-IL-15 showed much weaker binding with splenic NK and NKT cells than sIL-15-Fc (FIG. 12). Altogether, pro-IL-15 induced much less peripheral lymphocytes expansion and less toxicity, compared to sIL-15-Fc.

### Pro-IL15 Preserved Antitumor Activity

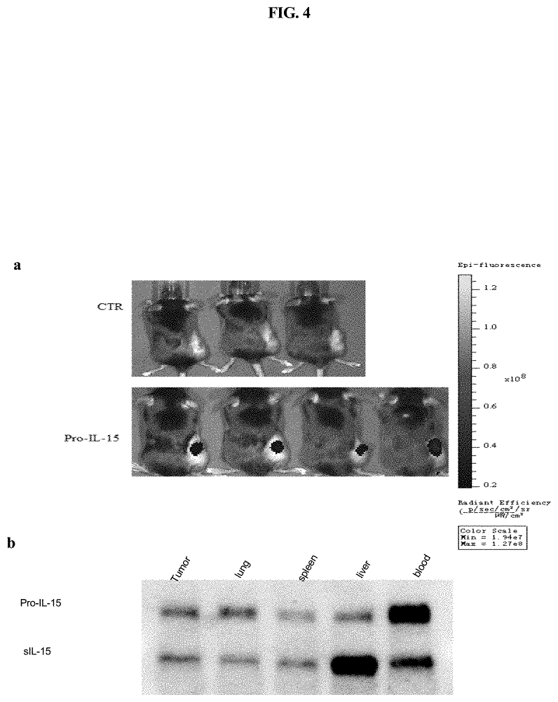

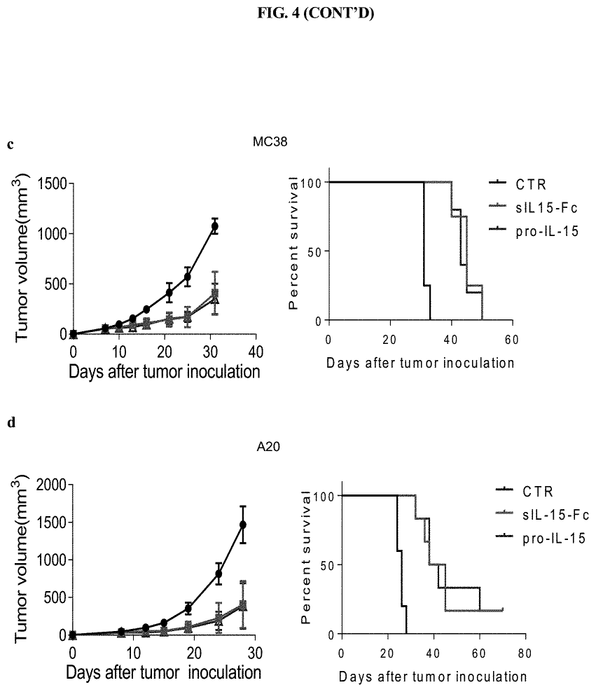

The expansion of lymphocytes within tumor tissues is critical to inhibit and eradicate tumor. We next sought to explore whether pro-IL-15 still target and inhibit tumor growth. The bioluminescence data showed that pro-IL-15 could accumulate within tumor tissue (FIG. 4A). Compared with blood, Pro-IL15 protein was digested preferentially within tumor tissue (FIG. 4B). Next, we compared the anti-tumor effect. Mice bearing MC38 or A20 tumor were treated with sIL-15-Fc or pro-IL-15 protein through i.v. injection. As showed, both the two proteins could efficiently inhibit tumor growth, and prolonged the mice survival (FIG. 4C and FIG. 4D). These data demonstrate that pro-IL-15 could accumulate and be activated within tumor tissues, and efficiently inhibit tumor growth.

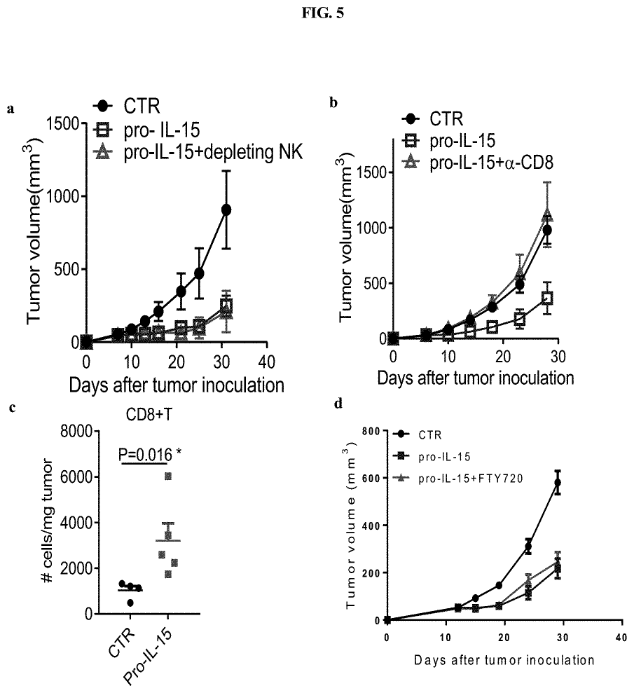

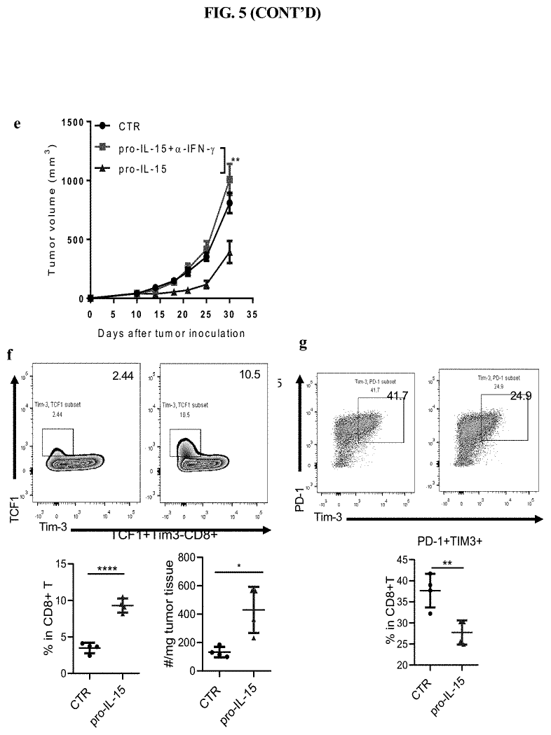

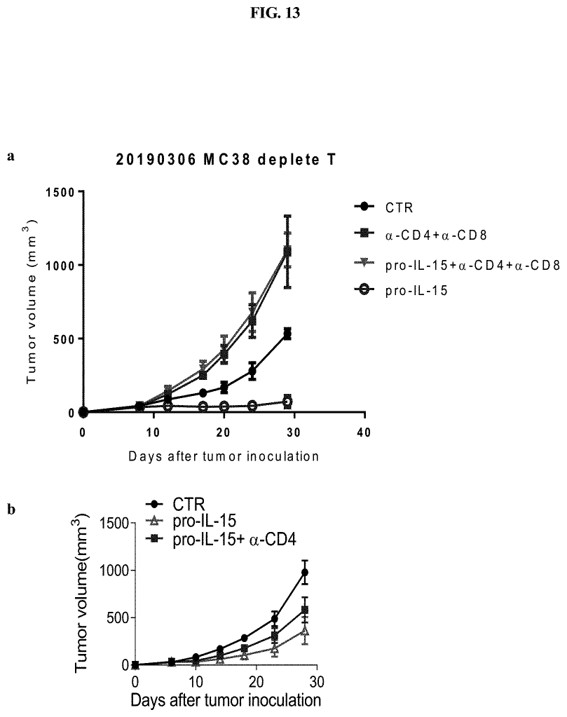

To test which cells contribute to pro-IL-15 mediate solid tumor control, we deplete different immune cells separately by antibodies. When NK or NKT cells are depleted, the anti-tumor effect of pro-IL-15 was not impaired (FIG. 5A). In contrast, depletion of CD4⁺ T and CD8⁺ T cells completely abrogated the anti-tumor effect of pro-IL-15, suggesting T lymphocytes are essential (FIG. 13A). Depletion of CD4⁺ T cells alone did not affect the tumor inhibition by pro-IL-15, suggesting CD4⁺ T cells are not essential (FIG. 13B). Further studies showed that CD8⁺ T cells are essential to mediate IL-15 mediated tumor control (FIG. 5B). We further checked the quantity change of CD8⁺ T cells within tumor tissue after pro-IL-15 treatment. The result showed that pro-IL-15 significantly increased the effector CD8⁺ T cells in tumor tissues (FIG. 5C). We next want to determine whether the increased CD8⁺ T cells within tumor is from expansion of pre-existing T cell inside tumor, or newly activated T cells migrating from peripheral lymphoid tissues into tumor. When utilizing FTY720 to block peripheral lymphocytes migrate into tumor tissue, pro-IL-15 still preserve the anti-tumor ability similar as non-FTY720 treated group (FIG. 5D). These data suggested that pro-IL-15 expand tumor-specific CD8⁺ T cells, which is essential and sufficient to inhibit the tumor growth. IFN-γ acts as one of the effector cytokines of T cells. To test whether IFN-γ contribute to pro-IL-15 mediate solid tumor control, mice were treated with anti-IFN-γ blocking antibody during pro-IL-15 treatment, which resulted in completely abrogated the antitumor effects, suggesting an essential role of IFN-γ. Recent study showed that TCF1⁺TIM3⁺ T cells within tumor showed stem-like phenotype and expand more efficiently. After pro-IL-15 treatment, both the percentage and quantity of the stem-like CD8⁺ T cells increased within tumor tissue (FIG. 5F). While the terminal exhausted CD8⁺ T (PD-1⁺Tim3⁺) cells decreased (FIG. 5G). These results showed that pro-IL-15 expand stem-like CD8⁺ T cells and decreased the terminal exhausted CD8⁺ T cells within tumor.

Pro-IL-15 Synergizes with Checkpoint Blockade to Control Advanced Tumor

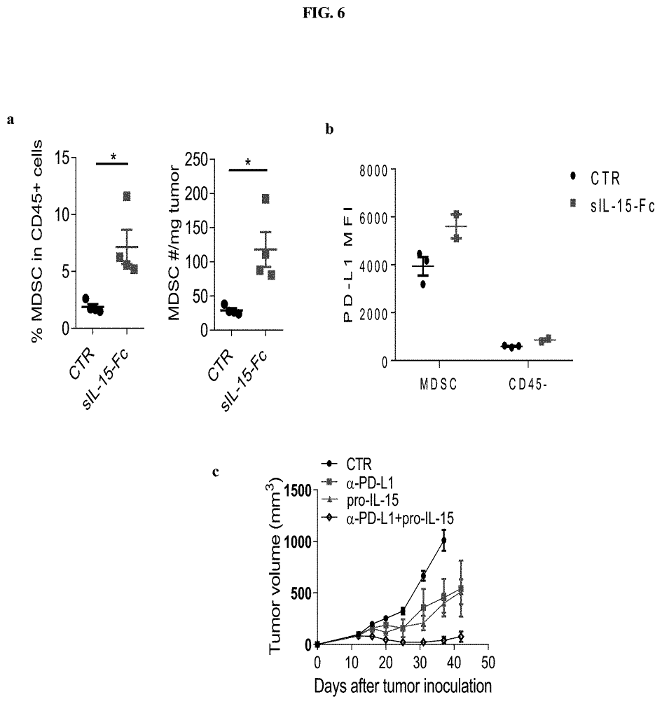

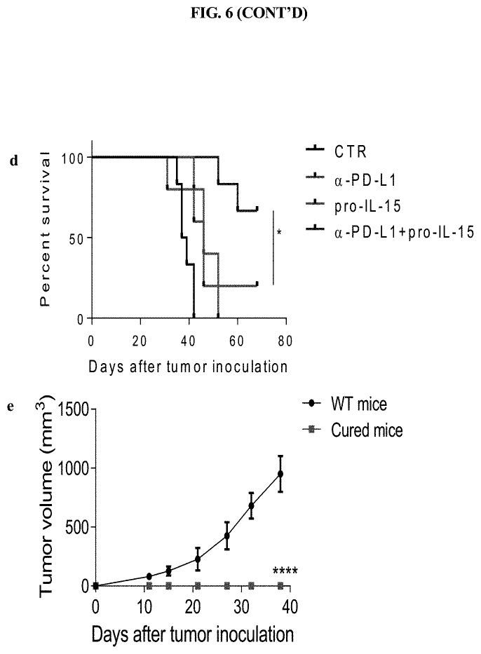

IL-15 treatment alone showed limited anti-tumor effect in both animal model and clinic, suggesting other potential immune inhibitory mechanism exists. After IL-15 treatment, we observed that both the percent and the absolute number of myeloid MDSCs were found notable increased (FIG. 6A). As myeloid cell within tumor tissues are major cell subsets expressing high PD-L1 molecule, we then checked PD-L1 expression profile. After IL-15 treatment, the expression of PD-L1 in MDSCs was highly upregulated (FIG. 6B). CD45⁻ tumor cells express much lower level of PD-L1 than MDSC myeloid derived suppressive (MDSC) cells after treatment of IL-15 (FIG. 6B). We speculated that blockade of PD-L1 could improve tumor-specific T cell response, and additional anti-PD-L1 antibody with IL-15 could achieve amplified anti-tumor effect. To test this hypothesis, MC38 tumor bearing mice were started treatment with pro-IL-15 or anti-PD-L1 from day 12, when tumor was well established. Either anti-PD-L1 or pro-IL-15 could only partially control the tumor growth, but the combination significantly enhanced the tumor control (FIG. 6C). During the combination treated group, 60% of mice achieved tumor-free survival eventually, and only 10% of mice in the single treatment group were tumor free (FIG. 6D). We next explored that whether the combination of anti-PD-L1 and pro-IL-15 treatment could help establish prolonged protective immunity and prevent the tumor relapse, which is valuable in clinic. Mice with complete tumor regression after combination therapy were re-challenged with lethal dose of MC38 cells. All of the previous tumor cleared mice rejected the re-challenged tumor, showing a strong memory response (FIG. 6E).

### Materials and Methods

### Mice

Female (6-8 weeks old) BALB/c and C57BL/6 mice were purchased from Vital River Laboratories (Beijing, China). All mice were maintained under specific pathogen-free (SPF) conditions in the animal facility of the Institute of Biophysics. Animal care and experiments were performed in accordance with the guidelines of the Institute of Biophysics, Chinese Academy of Sciences, using protocols approved by the Institutional Laboratory Animal Care and Use Committee.

### Cell Lines and Reagents

The 293F cells were kindly provided by Dr. Ting Xu (Alphamab, Suzhou, Jiangsu, China) and grown in SMM 293-TI medium (M293TI, Sino Biological). A20 and MC38 cell lines were purchased from ATCC (Manassas, Va.). MC38 was cultured in 5% CO₂ and maintained in vitro in Dulbecco's modified Eagle's medium, supplemented with 10% heat-inactivated fetal bovine serum, 2 mmol/l L-glutamine, 0.1 mmol/1 Minimum Essential Medium nonessential amino acids, 100 U/ml penicillin, and 100 mg/ml streptomycin. A20 cells and CTLL-2 cells were maintained in vitro in RPMI 1640 medium. Anti-PD-L1 Ab (10F.9G2) and anti IFN-γ Ab (R4-6A2) were purchased from BioXCell (West Lebanon, N.H.). Anti-CD8 Ab (TIB210), anti-CD4 Ab (GK1.5), FcγRII/III blocking Ab (2.4G2) and anti-NK1.1 Ab (PK136) were produced in house. Anti-Asialo-GM1 Ab (Poly21460) was purchased from Biolegend. FTY720 was purchased from Sigma.

### Production of IL-15 and Pro-IL-15 Fusion Protein

IL-15-Rα-Fc: the cDNA encoding mouse IL-15 mature mouse and IL-15Rα-sushi domain (amino acids 1-78) sequence was fused by a 26-amino acid linker (SGGGSGGGGSGGGGSGGGGSGGGSLQ). The signal peptide of human IgGκ leading sequence and hIgG1 Fc was used. The entire sequence was then cloned into the pEE12.4 vector (Lonza). Pro-IL-15: the cDNA encoding mouse IL-15Rβ EDC or IL-15RβD1 and IL-15-Rα-Fc was fused by a 20-amino acid linker (GGGGSSGFIANPVTAGGGGS). The entire sequence was then cloned into the pEE12.4 vector (Lonza). The plasmid was transiently transfected into 293F cells. Supernatants were collected on day 7 after transfection. The fusion protein was purified using a Protein A-Sepharose column according to the manual (Repligen Corporation).

### Toxicity

C57BL/6 mice bearing subcutaneous MC38 tumors were treated with lethal dose of sIL-15-Fc (80 μg or 0.8 μmol) or comparable molecular of pro-IL-15 by intravenously injection on day 8 and 11 after tumor inoculation. The body weight change of mice was monitored. The level of ALT and AST in the peripheral serum was quantified at day 1 and 4 after the first treatment. Cytokine levels in the serum which was collected at 24 hours after the first treatment were measured by cytometric bead array(CBA). Four days after the first treatment, mice were sacrificed and liver tissues were collected for HE staining.

### In Vitro Digestion Conditions for Pro-IL15

rhMMP-14 (R&D Systems) was activated with rhFurin in Activation Buffer (50 mM Tris, 1 mM CaCl₂), 0.5% (w/v) Brij-35, pH 9.0) at 37° C. for 2 hours. Pro-IL-15 was co-cultured with activated MMP14 in Assay Buffer (50 mM Tris, 3 mM CaCl₂), 1 μM ZnCl₂, pH 7.5) at 37° C. for 12 hours. The proteins cleavage was identified by reduced SDS-PAGE and released activity of pro-IL-15 was detected by CTLL-2 proliferation assay.

### CTLL-2 Proliferation Assay

Functional IL-15 was measured using CTLL-2 cells. 100 μL purified proteins or MMP cleaved products which were serially diluted were added per well to a 96-well plate, then 3000 CTLL-2 cells in 100 μL medium were added and incubated for 72 hours at 37° C. in 5% CO₂. 20 μL CCK8 (Cell Counting Kit-8) was added and the plate was incubated for 3-4 hours at 37° C. in 5% CO₂. Absorbance was read at 450 nm.

### Fluorescence Imaging

Pro-IL-15 was labeled with Cy5.5, purified. 25 μg Fluorescently-labeled Pro-IL-15 was injected intravenously into C57BL/6 mice bearing subcutaneous MC38 tumors. Fluorescence was measured with IVIS Spectrum at different time points.

### Flow Cytometry

Tumor tissues were collected, cut into small pieces, and re-suspended in digestion buffer (RPMI-1640 medium with 1 mg/ml type IV collagenase and 100 μg/mL DNase I). Tumors were digested for 45 min at 37° C., then passed through a 70 μm cell strainer to make single cell suspensions. Cells suspended in FACS buffer (1% bovine serum albumin and 0.05% NaN₃) were blocked with anti-CD16/32 Ab (anti-FcγIII/II receptor, clone 2.4G2) for 30 min and then stained with specific antibodies for 30 min on ice. For intracellular TCF-1 staining, samples were fixed, permeabilized, and stained with anti-mouse TCF-1. All fluorescent-labeling mAbs were purchased from BioLegend or eBioscience. DAPI or LIVE/DEAD™ fixable yellow dye (ThermoFisher) was used to exclude dead cells. Samples were analyzed on a FACSCalibur or Fortessa flow cytometer (BD Biosciences). Data were analyzed using FlowJo software (TreeStar).

### Tumor Growth and Treatments

Approximately 2-5×10⁵ of MC38 was injected subcutaneously into the right flank of C57BL/6 mice. A20 cells (3×10⁶) were subcutaneously injected into the right flank of Balb/c mice. Tumor volumes were measured and calculated (length×width×height/2). After the tumor was established, mice were treated with sIL15-Fc or pro-IL-15. For depletion of different types of cells, anti-CD8 Ab (clone TIB210) or anti-CD4 Ab (clone GK1.5) was injected i.p. at a dose of 200 μg on the same day of pro-IL-15 or sIL-15-Fc treatment and every 4 days thereafter. Anti-NK1.1 Ab (clone PK136) was injected i.p. at a dose of 400 μg on the same day of pro-IL-15 or sIL-15-Fc treatment and every 4 days thereafter. Anti-Asialo GM1 Ab was injected i.p. at a dose of 20 μl on the same day of pro-IL-15 or sIL-15-Fc treatment and every 4 days thereafter. For blocking IFN-γ, Anti-IFN-γ Ab (clone R4-6A2) was injected i.p. at a dose of 500 μg on the same day of pro IL-15 treatment and every 4 days thereafter. To block lymphocyte trafficking, mice were intraperitoneally injected with 25 μg FTY720, and 20 μg of FTY720 was administered every other day to maintain the blockade.

### Statistical Analysis

Data are shown as the mean±SEM. Statistical analyses were compared using an unpaired Student's two-tailed t test. Analyses were performed using GraphPad Prism version 5.0 (GraphPad Software). Statistically significant differences of p<0.05, p<0.01 and p<0.001 are noted with *, ** and ***, respectively.

SEQ Listing

SEQ ID No. 1: hIL15
NWVNVISDLKKIEDLIQSMHIDATLYTESDVHPSCKVTAMKCFLLELQVISLESGDASIHDTVENLIILANNSLSSNGNVTESGCKECEE
LEEKNIKEFLQSFVHIVQMFINTS
SEQ ID No. 2: hIgG1Fc (Fcdw)
EPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQD
WLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKL
TVDKSRWQQGNVFSCSVMHEALHNHYTQKSLSLSPGK
SEQ ID No. 3: hIgG1Fcmutant (Fcdm)
EPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQ
DWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSK
LTVDKSRWQQGNVFSCSVMHEALHNHYTQKSLSLSPGK
SEQ ID No. 4: hRa (sushi domain)
ITCPPPMSVEHADIWVKSYSLYSRERYICNSGFKRKAGTSSLTECVLNKATNVAHWTTPSLKCIRDPALVHQRPAPP
SEQ ID No. 5: hRb (27 aa~240 aa)
AVNGTSQFTCFYNSRANISCVWSQDGALQDTSCQVHAWPDRRRWNQTCELLPVSQASWACNLILGAPDSQKLTTVDIVTLRVLCR
EGVRWRVMAIQDFKPFENLRLMAPISLQVVHVETHRCNISWEISQASHYFERHLEFEARTLSPGHTWEEAPLLTLKQKQEWICLETL
TPDTQYEFQVRVKPLQGEFTTWSPWSQPLAFRTKPAALGKDT
SEQ ID No. 6: hRb (27 aa~234 aa)
AVNGTSQFTCFYNSRANISCVWSQDGALQDTSCQVHAWPDRRRWNQTCELLPVSQASWACNLILGAPDSQKLTTVDIVTLRVLCR
EGVRWRVMAIQDFKPFENLRLMAPISLQVVHVETHRCNISWEISQASHYFERHLEFEARTLSPGHTWEEAPLLTLKQKQEWICLETL
TPDTQYEFQVRVKPLQGEFTTWSPWSQPLAFRTKPA
SEQ ID No. 7: hRb (32 aa~240 aa)
SQFTCFYNSRANISCVWSQDGALQDTSCQVHAWPDRRRWNQTCELLPVSQASWACNLILGAPDSQKLTTVDIVTLRVLCREGVR
WRVMAIQDFKPFENLRLMAPISLQVVHVETHRCNISWEISQASHYFERHLEFEARTLSPGHTWEEAPLLTLKQKQEWICLETLTPDT
QYEFQVRVKPLQGEFTTWSPWSQPLAFRTKPAALGKDT
SEQ ID No. 8: hRb (32 aa~234 aa)
SQFTCFYNSRANISCVWSQDGALQDTSCQVHAWPDRRRWNQTCELLPVSQASWACNLILGAPDSQKLTTVDIVTLRVLCREGVR
WRVMAIQDFKPFENLRLMAPISLQVVHVETHRCNISWEISQASHYFERHLEFEARTLSPGHTWEEAPLLTLKQKQEWICLETLTPDT
QYEFQVRVKPLQGEFTTWSPWSQPLAFRTKPA
SEQ ID No. 9: hRb (27 aa~125 aa)
AVNGTSQFTCFYNSRANISCVWSQDGALQDTSCQVHAWPDRRRWNQTCELLPVSQASWACNLILGAPDSQKLTTVDIVTLRVLCR
EGVRWRVMAIQDFK
SEQ ID No. 10:: hRb (27 aa~128 aa)
AVNGTSQFTCFYNSRANISCVWSQDGALQDTSCQVHAWPDRRRWNQTCELLPVSQASWACNLILGAPDSQKLTTVDIVTLRVLCR
EGVRWRVMAIQDFKPFE
SEQ ID No. 11: hRb (27 aa~137 aa)
AVNGTSQFTCFYNSRANISCVWSQDGALQDTSCQVHAWPDRRRWNQTCELLPVSQASWACNLILGAPDSQKLTTVDIVTLRVLCR
EGVRWRVMAIQDFKPFENLRLMAPIS
SEQ ID No. 12: hRb (32 aa~137 aa)
SQFTCFYNSRANISCVWSQDGALQDTSCQVHAWPDRRRWNQTCELLPVSQASWACNLILGAPDSQKLTTVDIVTLRVLCREGVR
WRVMAIQDFKPFENLRLMAPIS
SEQ ID No. 13: hRb (83 aa~214 aa)
SQASWACNLILGAPDSQKLTTVDIVTLRVLCREGVRWRVMAIQDFKPFENLRLMAPISLQVVHVETHRCNISWEISQASHYFERHLE
FEARTLSPGHTWEEAPLLTLKQKQEWICLETLTPDTQYEFQVRVKPLQ
SEQ ID No. 14: hRb (83 aa~234 aa)
SQASWACNLILGAPDSQKLTTVDIVTLRVLCREGVRWRVMAIQDFKPFENLRLMAPISLQVVHVETHRCNISWEISQASHYFERHLE
FEARTLSPGHTWEEAPLLTLKQKQEWICLETLTPDTQYEFQVRVKPLQGEFTTWSPWSQPLAFRTKPA
SEQ ID No. 15: hRb (83 aa~240 aa)
SQASWACNLILGAPDSQKLTTVDIVTLRVLCREGVRWRVMAIQDFKPFENLRLMAPISLQVVHVETHRCNISWEISQASHYFERHLE
FEARTLSPGHTWEEAPLLTLKQKQEWICLETLTPDTQYEFQVRVKPLQGEFTTWSPWSQPLAFRTKPAALGKDT
SEQ ID No. 16: L1: Non-cleavable linker. n = 1~10
L1.1: (G2S)n;
L1.2: (G3S)n;
L1.3: (G4S)n;
L1.4: SGGGSGGGGS GGGGSGGGGS GGGSLQ
SEQ ID No. 17:,: short cleavable linkers(L2), including:,
L2.0: L1-SGRSPAIFTA-L1
L2.1: L1-SGRSENIRTA-L1
L2.2: L1-SGARYRWLTA-L1
L2.3: L1-SGRSYAILTA-L1
L2.4: L1-SGRAMHMYTA-L1
L2.5: L1-SPVGLIG-L1
L2.6: L1-SGRIGFLRTA-L1
L2.7: L1-SGAIGFLRTA-L1
L2.8: L1-SGAAMHMYTA-L1
L2.9: L1-SGASENIRTA-L1
L2.10: L1-SGRPENIRTA-L1
L2.11: L1-SGAPENIRTA-L1
L2.12: L1-SGLISHSITA-L1
L2.13: L1-SGNLRSKLTA-L1
L2.14: L1-SGVFSIPLTA-L1
L2.15: L1-SGIKYHSLTA-L1
SEQ ID No. 18:: long cleavable linkers (L2L),composed of following two short linkers as examples,
but not excluding others combinations:
L2L.0.1 L1-SGRSPAIFTA-L1-SGRSENIRTA-L1
L2L.1.0 L1-SGRSENIRTA-L1-SGRSPAIFTA-L1
L2L.2.1 L1-SGARYRWLTA-L1-SGRSENIRTA-L1
L2L.1.2 L1-SGRSENIRTA-L1-SGARYRWLTA-L1
L2L.4.1 L1-SGRAMHMYTA-L1-SGRSENIRTA-L1
L2L.1.4 L1-SGRAMHMYTA-L1-SGRAMHMYTA-L1
L2L.0.2 L1-SGRSPAIFTA-L1-SGARYRWLTA-L1
L2L.2.0 L1-SGARYRWLTA-L1-SGRSPAIFTA-L1
L2L.0.3 L1-SGRSPAIFTA-L1-SGRSYAILTA-L1
L2L.3.0 L1-SGRSYAILTA-L1-SGRSPAIFTA-L1
L2L.0.4 L1-SGRSPAIFTA-L1-SGRAMHMYTA-L1
L2L.4.0 L1-SGRAMHMYTA-L1-SGRSPAIFTA-L1
L2L.2.3 L1-SGARYRWLTA-L1-SGRSYAILTA-L1
L2L.3.2 L1-SGRSYAILTA-L1-SGARYRWLTA-L1
L2L.2.4 L1-SGARYRWLTA-L1-SGRAMHMYTA-L1
L2L.4.2 L1-SGRAMHMYTA-L1-SGARYRWLTA-L1
L2L.3.4 L1-SGRSYAILTA-L1-SGRAMHMYTA-L1
L2L.4.3 L1-SGRAMHMYTA-L1-SGRSYAILTA-L1
SEQ ID No. 19: CH1:
ASTKGPSVFPLAPSSKSTSGGTAALGCLVKDYFPEPVTVSWNSGALTSGVHTFPAVLQSSGLYSLSSVVTVPSSSLGTQTYICNVN
HKPSNTKVDKRVEPKSC
SEQ ID No. 20: CL
RTVAAPSVFIFPPSDEQLKSGTASVVCLLNNFYPREAKVQWKVDNALQSGNSQESVTEQDSKDSTYSLSSTLTLSKADYEKHKVYA
CEVTHQGLSSPVTKSFNRGEC
Schematic drawings:
Model A:
1100: control
hIL15-Ra-L1-Fc(dw,dm)
1023, 1102, 1172, 1188 (L2.0) 1191 (L2L)
Rb(DM1~2)-L2-hIL15-L1-Ra-L1-Fc(dw, dm)
Rb(DM1~2)-L2L-hIL15-L1-Ra-L1-Fc(dw,dm)
Model B:
IL15-CH1-L1-Fc(dw,dm) + Ra-CL (control)
Rb(DM1~2)-L2 or L2L-IL15-L1-CH1-Fc(dw,dm) + Ra-CL

Applicant's disclosure is described herein in preferred embodiments with reference to the Figures, in which like numbers represent the same or similar elements. Reference throughout this specification to “one embodiment,” “an embodiment,” or similar language means that a particular feature, structure, or characteristic described in connection with the embodiment is included in at least one embodiment of the present invention. Thus, appearances of the phrases “in one embodiment,” “in an embodiment,” and similar language throughout this specification may, but do not necessarily, all refer to the same embodiment.

The described features, structures, or characteristics of Applicant's disclosure may be combined in any suitable manner in one or more embodiments. In the description, herein, numerous specific details are recited to provide a thorough understanding of embodiments of the invention. One skilled in the relevant art will recognize, however, that Applicant's composition and/or method may be practiced without one or more of the specific details, or with other methods, components, materials, and so forth. In other instances, well-known structures, materials, or operations are not shown or described in detail to avoid obscuring aspects of the disclosure.

Unless defined otherwise, all technical and scientific terms used herein have the same meaning as commonly understood by one of ordinary skill in the art. Although any methods and materials similar or equivalent to those described herein can also be used in the practice or testing of the present disclosure, the preferred methods and materials are now described. Methods recited herein may be carried out in any order that is logically possible, in addition to a particular order disclosed.

### INCORPORATION BY REFERENCE

References and citations to other documents, such as patents, patent applications, patent publications, journals, books, papers, web contents, have been made in this disclosure. All such documents are hereby incorporated herein by reference in their entirety for all purposes. Any material, or portion thereof, that is said to be incorporated by reference herein, but which conflicts with existing definitions, statements, or other disclosure material explicitly set forth herein is only incorporated to the extent that no conflict arises between that incorporated material and the present disclosure material. In the event of a conflict, the conflict is to be resolved in favor of the present disclosure as the preferred disclosure.

### EQUIVALENTS

The representative examples are intended to help illustrate the invention, and are not intended to, nor should they be construed to, limit the scope of the invention. Indeed, various modifications of the invention and many further embodiments thereof, in addition to those shown and described herein, will become apparent to those skilled in the art from the full contents of this document, including the examples and the references to the scientific and patent literature included herein. The examples contain important additional information, exemplification and guidance that can be adapted to the practice of this invention in its various embodiments and equivalents thereof.

1. A fusion protein or fusion protein complex (A), being a homodimeric complex and comprising:
a first structural unit: a whole or a subunit domain of the interleukin 15 (IL15) receptor-α;
a second structural unit: an active IL15;
a third structural unit located at the C-terminus of the fusion protein: an antibody Fc fragment;
a fourth structural unit located at the N-terminus of the fusion protein: a whole or a subunit domain of the IL15 receptor β linked to the first, second or third structure unit via a short cleavable linker (L) or long cleavable linker (L2L); and
one or more connecting segments or ligation fragments as flexible non-cleavable linkers (L1) for connecting the different units.

2. A fusion protein or fusion protein complex (B), being a homodimeric complex and comprising:
a first fragment (Fragment No. 1), comprising:
a first structural unit: a whole or a subunit domain of the interleukin 15 (IL15) receptor-α, and
a fifth structural unit: a constant region of light chain (CL); and
a second fragment (Fragment No. 2), comprising:
a second structural unit: an active IL15,
a third structural unit located at the C-terminus of the fusion protein: an antibody Fc fragment,
a fourth structural unit located at the N-terminus of the fusion protein: a whole or a subunit domain of the IL15 receptor β; and linked with either short cleavable linker (L2) or long cleavable linker (L2L),
a sixth structural unit: a constant region of heavy chain (CH), and
one or more connecting segments or ligation fragments as flexible non-cleavable linkers (L1) for connecting the different units,
wherein two Fragment No. 1 and two Fragment No. 2 are joined together to form a complex via disulfide bonds between CH and CL and between two Fc's.

3. The fusion protein or fusion protein complex of claim 1 & or 2, wherein the active IL15 is a human IL15 and the antibody Fc fragment is a human Fc.

4. The fusion protein or fusion protein complex of claim 3, wherein the first structural unit is a whole interleukin 15 (IL15) receptor-α.

5. The fusion protein or fusion protein complex of claim 3, wherein the first structural unit is a subunit domain of interleukin 15 (IL15) receptor-α.

6. The fusion protein or fusion protein complex of claim 5, wherein the fourth structural unit is a subunit domain of the IL15 receptor β having less than the whole length 27aa-240aa of IL15 receptor β.

7. The fusion protein or fusion protein complex of claim 5, wherein the subunit domain of the IL15 receptor β is selected from a shortened IL15 receptor β represented by 27aa-125aa, 27aa-128aa, 27aa˜137aa, 32aa-234aa, 32aa-137aa, 32aa-240aa, and 83aa-214aa.

8. The fusion protein or fusion protein complex of claim 6, wherein the subunit domain of the IL15 receptor β is a shortened IL15 receptor β represented by 27-125aa.

9. The fusion protein or fusion protein complex of claim 3, having a construct as shown in FIG. 14A.

10. The fusion protein or fusion protein complex of claim 3, having a construct as shown in FIG. 14B.

11. The fusion protein or fusion protein complex of claim 3, wherein one of the connecting segments or ligation fragments comprises an amino acid sequence that is single or a multiple of GGGGS or (G)ₙS, n=2˜4 with or without protease sensitive sequences.

12. The fusion protein or fusion protein complex of claim 3, comprising a short cleavable flexible linker (L2).

13. The fusion protein or fusion protein complex of claim 3, comprising a long cleavable flexible linker (L2L).

14. The fusion protein or fusion protein complex of claim 12 or 13, wherein the short cleavable flexible linker (L2) and the long cleavable flexible linker (L2L) are capable of being recognized and hydrolyzed by a proteolytic enzyme specifically expressed in a tumor microenvironment.

15-17. (canceled)

18. A prodrug comprising a fusion protein or fusion protein complex of claim 3.

19. A substantially purified fusion protein or fusion protein complex, or prodrug thereof, of claim 3.

20. A polynucleotide encoding a fusion protein or fusion protein complex, or prodrug thereof, of claim 3.

21. An expression vector comprising the polynucleotide of claim 20.

22. A pharmaceutical composition comprising a fusion protein or fusion protein complex, or prodrug thereof, of claim 3.

23. A method for treating a disease or condition, comprising:
administering to a patient in need thereof a therapeutically effective amount of a fusion protein or fusion protein complex, or prodrug thereof, of claim 3,
wherein the disease or condition is selected from hyperplasia, solid tumor or hematopoietic malignancy.

24-34. (canceled)
